(19) United States
(12) Patent Application Publication
(10) Pub. No.: US 2025/0269095 A1
(43) Pub. Date: Aug. 28, 2025

(54) METHODS OF CHARACTERIZING A BIOACTIVE COATED PRODUCT

(71) Applicant: Riverpoint Medical, LLC, Portland, OR (US)

(72) Inventors: Edwin K. Anderson, Ridgefield, WA (US); Douglas S. Rowley, Portland, OR (US)

(21) Appl. No.: 19/195,690

(22) Filed: Apr. 30, 2025

Related U.S. Application Data
(63) Continuation of application No. PCT/US2023/036249, filed on Oct. 30, 2023.
(60) Provisional application No. 63/492,027, filed on Mar. 24, 2023.
(60) Provisional application No. 63/381,650, filed on Oct. 31, 2022.

(51) Int. Cl. A61L 31/08; A61L 17/14; A61L 31/06; G01N 23/20091; G01N 33/36

(52) U.S. Cl. CPC A61L 31/086; A61L 17/145; A61L 31/06; G01N 23/20091; G01N 33/36

(57) ABSTRACT

The disclosed inventions relate to an improved bioceramic coated product and methods of characterizing coated products. More specifically, the disclosed inventions relate to an improved bioceramic coated product comprising bioceramic particles that has a homogeneous or uniform application that should reliably provide consistent healing response properties after implantation. The consistent healing responses may include enhanced osteoconductive properties or bone growth, antimicrobial activity, and/or angiogenic activity.

### CROSS-REFERENCE TO RELATED APPLICATIONS

This application is a continuation of International PCT Application No. PCT/US2023/036249, filed on Oct. 30, 2023, entitled “Methods of Characterizing a Bioactive Coated Product,” which claims the benefit of U.S. Provisional Appl. No. 63/492,027, filed on Mar. 24, 2023, entitled “Apparatus and Characterization Methods of a Bioactive Coated Product,” and U.S. Provisional Appl. No. 63/381,650, filed on Oct. 31, 2022, entitled “Apparatus and Characterization Methods of a Bioactive Coated Product,” and are incorporated by reference in their entireties.

### TECHNICAL FIELD

The disclosed inventions relate to an improved coated product and methods of characterizing coated products. More specifically, the disclosed inventions relate to one or more characterization methods used to generate a set of quality control (QC) inspection criteria that can reproducibly manufacture a homogeneously coated product comprising at bioactive ceramic particles to provide a consistent and optimal healing response properties after implantation.

### BACKGROUND OF THE INVENTION

Uncoated textiles constructs made from fibers or filaments spun from synthetic polymers have found various applications as a component of a medical device, such as in surgical sutures and cables, artificial ligaments and tendons, hernia meshes, and flexible tissue anchors. The attachment of the uncoated textiles to bone is typically obtained by inserting it into a hole drilled in the bone (also called bore or tunnel) and connecting to soft tissue via a suture attached to the anchor. Consequently, uncoated textile constructs and/or tissue anchors are made from PET or other synthetic polymers that are generally bioinert and do not intrinsically bind to bone. Since the unmodified bioinert orthopedic implants and/or PET based orthopedic implants do not bind to bone, they elicit a poor healing or excessive healing response and become prone to fibrous tissue encapsulation after implantation in the bore or bone tunnels. Without strong bonding between host bone and implant (e.g., osteointegration), continuously changing loads, and/or micromotion of the unmodified implants may lead to enlargement of the bore or bone tunnels causing instability, implant loosening, implant migration, implant detachment and/or cyst formation (see e.g., Pfeiffer et al, DOI: 10.1016/j.jse.2013.12.036).

As a result, bioactive ceramics are promising biomaterials that can be successfully applied as coatings on the surface of different flexible or rigid constructs. Bioactive ceramic coatings are shown to bond to both hard and soft tissues stimulating cells towards a path of regeneration and self-repair. Unfortunately, there have been some challenges with the bioactive ceramics as coatings, such as poor adherence at the interface, coating consistency, contamination, etc., leading to non-induction of the expected biocompatible behavior and relevant tissue/cell reactions.

### BRIEF SUMMARY OF THE INVENTION

Therefore, a need exists to characterize and manufacture a bioactive coated product in order implant to develop one or more quality control inspections criteria of the bioactive coating and manufacture implants that meet these QC inspection criteria. The QC inspection criteria is intended to provide a manufacturer with a repeatable process that creates a homogeneously bioactive coated product to accomplish good bone or tissue bonds and consistent, desired biological interactions. These desired interactions include a positive response through the formation of a strong tissue-implant bond and the genetic activation of specific cell pathways.

In one embodiment, a method of manufacturing a homogenously coated textile product, comprises the steps of: depositing a first coating layer onto a portion of a textile product, the first coating layer comprises a first solvent and a first material, the first material includes a biocompatible, nonbiodegradable polymer; depositing a second coating layer onto the first coating layer, the second coating layer comprises a second solvent and a second coating material, the second coating material includes one or more bioceramic particles having a plurality of molecules comprising at least Sodium (Na), Phosphorus (P), Silicone (Si) and Calcium (Ca); removing the second solvent from the second coating layer to enable at least a portion of the one or more bioceramic particles to be positioned below an outer surface of the first coating layer; and verifying that the coated textile product comprises a homogeneous second coating layer by meeting or exceeding the homogeneity osteoconductive (OC) ratio of 4.7 wt %, wherein the homogeneity OC ratio equals the sum of the wt % of the plurality of molecules of the one or more bioceramic particles of the second coating layer acquired by Energy Dispersive X-Ray Spectroscopy (EDS) on at least one predetermined location along a length of the coated textile product and divided by four.

The method of characterizing a homogeneously coated textile product, comprising the steps of: manufacturing at least one coated textile product; the at least one coated textile product comprises: (a) a textile product, the textile product comprising an outer surface and a product material, the product material includes a biocompatible, nonbiodegradable polymer; (b) a first coating, the first coating comprising a first coating material, the first coating disposed over a portion of the outer surface of the textile product, the first coating material includes a biocompatible, nonbiodegradable polymer; and (c) a second coating, the second coating disposed over a portion of the first coating, the second coating comprising one or more bioceramic particles, at least a portion of the one or more bioceramic particles is positioned below an outer surface of the first coating, the one or more bioceramic particles includes a plurality of elements of at least Sodium (Na), Phosphorus (P), Silicone (Si) and Calcium (Ca); collecting the wt % of each of the plurality of elements of the one or more bioceramic particles of the second coating from at least one predetermined location on the at least one coated textile product using Energy Dispersive X-Ray Spectroscopy (EDS); determining the homogeneity OC ratio by summing the wt % of each of the plurality of elements of the second coating and dividing by four; and confirming the homogeneity OC ratio meets or exceeds 4.7 wt %, wherein the homogeneity OC ratio of 4.7 wt % or greater produces a homogeneously coated textile product with a consistent in-vivo bioactive response.

A method of developing quality control (QC) acceptance criteria to manufacture bioceramic coated products having homogenous bioceramic coating comprises the steps of: obtaining at least one bioceramic coated product, the at least one bioceramic coated product comprising one or more bioceramic particles, the one or more bioceramic particles comprising a plurality of elements that include Sodium (Na), Phosphorus (P), Silicone (Si) and Calcium (Ca); characterizing the one or more bioceramic coated products by collecting the wt % of each of the plurality of elements of the one or more bioceramic particles of the from at least one predetermined location on the at least one bioceramic coated textile product using Energy Dispersive X-Ray Spectroscopy (EDS); determining an average wt % of the plurality of elements of the one or more bioceramic particles by summing the wt % of each of the plurality of elements of the one or more bioceramic particles of the at least one bioceramic coated product and dividing by four; and generating at least one QC acceptance criteria minimum value by applying a desired statistical model, the at least one QC acceptance criteria includes the homogeneity osteoconductive (OC) ratio, wherein the homogeneity OC ratio enables acceptance of a manufacturing process for the at one least coated product having a homogeneous bioceramic coating for consistent biological response after implantation.

### BRIEF DESCRIPTION OF THE SEVERAL VIEWS OF THE DRAWINGS

FIG. 1 illustrates a flowchart with one embodiment of a methods of quality control (QC) inspection of one or more coated products;

FIG. 2 illustrates a flowchart with one embodiment of a method of predefining a set of QC inspection criteria by characterizing the one or more coated products;

FIGS. 3A-3C illustrates tables describing different characterization techniques or methods for one or more bioactivity responses;

FIG. 3D illustrates a table describing various quality control acceptance criteria;

FIGS. 4A-4H depicts different SEM images of Sample 11.0 at different site locations and magnifications;

FIGS. 5A-5H depicts different SEM images of sample 11.5 at different site locations and magnifications;

FIGS. 6A-6H depicts different SEM images of sample 12.0 at different site locations and magnifications;

FIGS. 7A-7B depicts an SEM and EDS layered image of sample 11.0;

FIGS. 8A-8B depicts an SEM and EDS layered image of sample 11.5;

FIGS. 9A-9B depicts an SEM and EDS layered image of sample 12.0;

FIGS. 10A-10D depicts EDS color maps of samples 11.0, 11.5, 12.0, and DSM-2 for the CK functional group;

FIGS. 11A-11D depicts EDS color maps of samples 11.0, 11.5, 12.0, and DSM-2 for the OK functional group;

FIGS. 12A-12D depicts EDS color maps of samples 11.0, 11.5, 12.0, and DSM-2 for the FK functional group;

FIGS. 13A-13D depicts EDS color maps of samples 11.0, 11.5, 12.0, and DSM-2 for the Nak functional group;

FIGS. 14A-14D depicts EDS color maps of samples 11.0, 11.5, 12.0, and DSM-2 for the Sik functional group;

FIGS. 15A-15D depicts EDS color maps of samples 11.0, 11.5, 12.0, and DSM-2 for the PK functional group;

FIGS. 16A-16D depicts EDS color maps of samples 11.0, 11.5, 12.0, and DSM-2 for the CI-K functional group;

FIGS. 17A-17D depicts EDS color maps of samples 11.0, 11.5, 12.0, and DSM-2 for the Cak functional group;

FIGS. 18A-18D depicts EDS color maps of samples 11.0, 11.5, 12.0, and DSM-2 for the Ti-K functional group;

FIGS. 19A-19H depicts EDS Spectrum of Molecules Map of samples 11.0, 11.5, 12.0;

FIGS. 20A-20B depicts FTIR spectrum of molecules map of bioceramic coated products at baseline;

FIGS. 21A-21B depicts FTIR spectrum of molecules map of HAp at baseline;

FIGS. 22A-22C depicts FTIR spectrum of molecules map of Bionate at baseline;

FIGS. 23A-23B depicts FTIR spectrum of molecules map of Bioglass at baseline;

FIGS. 24A-24B depicts a data table and its respective bar graph of pull-out strength for bioceramic coated products and uncoated products;

FIGS. 25A-25B depicts histological images of Group 1, uncoated products, at 8 and 16 weeks, respectively;

FIGS. 26A-26B depicts histological images of Group 2, bioceramic coated products, at 8 and 16 weeks, respectively;

FIG. 27 depicts a graph comparing the linear relationship between the homogeneity OC ratio and bioceramic % coverage area;

FIG. 28A-28C depicts EDS color maps of bioceramic coated products;

FIGS. 29A-29B depicts SEM images and SEM-EDS spectrum maps for Group 1, uncoated products;

FIG. 29C shows a data table calculating the Group 1 homogeneity OC ratio;

FIGS. 30A-30B depicts SEM images and SEM-EDS spectrum maps for Group 2, bioceramic coated products; and

FIG. 30C shows a data table calculating the Group 2 homogeneity OC ratio.

### DETAILED DESCRIPTION OF THE INVENTION

There exists a need in industry for a coated textile product that can be manufactured to consistently and repeatably provide an improved, enhanced or optimized bioactive and biocompatibility response or properties (e.g., healing response), while preserving the mechanical properties needed to function in its intended medical application or have successful clinical outcomes. It is an object of this disclosure to provide (1) a coated textile product having homogeneous application of one or more bioactive coatings onto a surface; (2) a method to characterize one or more bioactive coated textile products; and (3) a method of creating quality control inspection criteria of the one or more bioactive coated textile products to ensure that the product contains sufficient coating to produce or enable an enhanced or consistent biocompatible and/or bioactive properties after implantation.

The improved or enhanced biocompatible and/or bioactive properties (e.g., healing response) includes an osteointegration or osteostimulation response. There are a wide variety of formulations that include varying elemental compositions or varying molecules. When bioactive ceramics are immersed in or exposed to physiological fluids, the bioactive ceramic “network” of elements degrades, and is subsequently released into the surrounding physiological fluid, where the process of hydroxyapatite (HA) layer formation begins. The HA layer formation plays a key role in the osteogenesis process or activity that results in the formation of strong bonds. The osteointegrative activity or response enables osseointegration after implantation, reduced implant enlargement of the bore or bone tunnel inner diameter, reducing instability, reducing implant loosening implant detachment, reducing implant migration, reducing cyst formation, improving immunological response at implantation site, improved pull-out strength at implantation site, reduced inflammation or inflammatory markers, reduced tissue reactions, reduced bone reactions, and/or any combination thereof.

Therefore, the “network” of elements released into the surrounding physiological fluid can dramatically impact the degradation rate, element release properties, osteointegration, antibacterial properties and/or any combination thereof. It important to establish a consistent and repeatable manufacturing process of bioceramic coated products and confirm the uniform or homogeneous application of the bioceramic coating onto the bioceramic coated product to accomplish good bone or tissue bonds and consistent, desired biological interactions.

A biocompatible material or compound herein means that any material or substance is biologically compatible by not producing a toxic, injurious, or immunologic response in living tissue. Accordingly, a biocompatible material or substance is a term to describe that the material or substance can exist in harmony with the body. However, bioactivity is the ability of a material to elicit a specific localized biological response at the interface of the material and cells, body fluid or tissue, due to its reactive surface. For example, osteoconductivity is a bioactivity that results in growth of bony tissue onto the surface or into the porous structure of an implant or graft. Osseointegration refers to the formation of a direct interface between an implant and bone tissue, without intervening soft tissue, and resulting in mechanical anchorage of the implant, i.e., the functional result of an osteoconductive implant. Osteogenesis is the formation of bone or development of bones, while osteoinduction refers to the act or process of stimulating osteogenesis. As previously disclosed herein, the improved or enhanced biocompatible and/or bioactive properties include the allowance of osseointegration after implantation, reduced implant enlargement of the bore or bone tunnel inner diameter, reducing instability, reducing implant loosening implant detachment, reducing cyst formation, improving immunological response at implantation site, improved pull-out strength at implantation site, reduced inflammation or inflammatory markers, reduced tissue reactions, and/or any combination thereof.

The improved or enhanced biocompatible and/or bioactive properties or response (e.g., healing response) include an antimicrobial response. Once bioactive glass is immersed in or exposed to physiological fluids, the bioactive glass leaches a network of ions (e.g., Na+, K+, Ca+ with H+) from the granules' surface resulting in the increase of osmotic pressure and pH, and the reduction the microbials' biofilm production. The releasing of the ions makes the surrounding environment hostile to microbial growth without affecting the host bone or tissue. The bioactive glass antimicrobial activity is effective against a wide selection of aerobic and anaerobic bacteria, either in planktonic or sessile forms, and/or antibiotic resistant microbes.

Although the following description is generally related to and illustrated with sutures, flexible tissue anchors and use thereof in bone, tendon and ligament reconstruction, it will be understood that the methods and articles disclosed herein can also be applicable to other rigid based products and related medical applications wherein improved bioactivity or biocompatible responses or properties (e.g., healing response) are desired.

In one embodiment, a coated textile product comprising: a textile and at least coating layer. The at least one coating layer comprises a bioceramic coating, the bioceramic coating including exposed ceramic particles adhered to it. The coated textile product can be used in a medical implant. The medical implant may be especially desired for use as an orthopedic implant for use in orthopedic surgery concerning the musculoskeletal system, which provides for form, stability, and movement of the body. This system is made up of the body's bones (the skeleton), muscles, cartilage, tendons, ligaments, joints, and other connective tissue (the tissue that supports and binds tissues and organs together). The coated textile product may comprise a suture, a bone anchor, plugs or screws, surgical meshes, vascular implants, bandages, wound care dressing, absorbents, and/or any combination thereof.

The musculoskeletal system's primary functions include supporting the body, allowing motion, and protecting vital organs. The joints and musculoskeletal tissues of the human body may be subject to traumatic injury, disease and degenerative processes that over a period of time can lead to the deterioration or failure of a joint causing severe pain or immobility. Generally, the ability of a joint to provide pain free articulation and carry load is dependent upon the presence of healthy bone, cartilage and associated musculoskeletal tissues that provide a stable joint. Examples of orthopedic implants include suture anchors, bone anchors, plugs and screws, which are used in repairing bone fractures or torn ligaments and tendons, or in securing implants like artificial ligaments, tendons or cartilage replacement devices to bone.

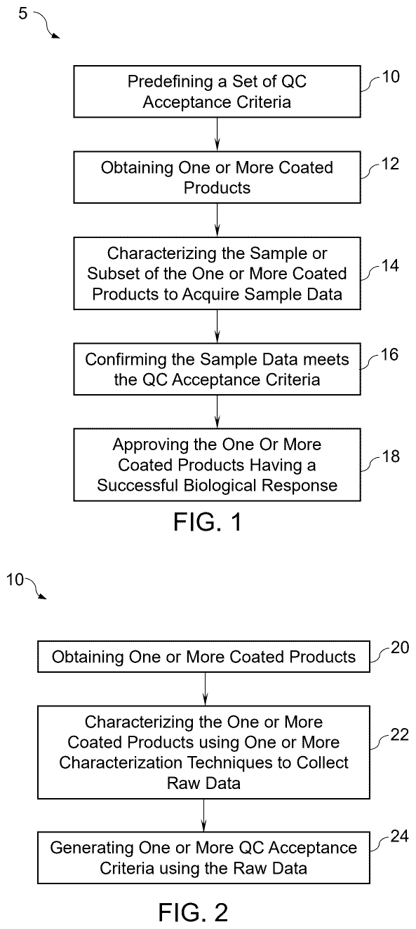

With reference to FIG. 1, the flowchart illustrates one embodiment of a method for quality control inspection of bioceramic coated products with consistent bioactive and/or biocompatible properties 5 comprises the steps of: predefining a one or more quality control (QC) inspection criteria that predicts or correlates one or more coated products having improved bioactive or biocompatible response 10; obtaining one or more coated products 12; characterizing the sample or subset of the one or more coated products to collect sample data 14; confirming the one or more coated products and/or sample or subset of the one or more coated products data meets or substantially meets one or more QC acceptance criteria 16; and approving the one or more coated products to be acceptable and have enhanced bioactive and/or biocompatible properties 18. Alternatively, FIG. 1 may be described as method for measuring, verifying or confirming bioceramic coating variability of manufactured coated products or a method for determining bioceramic coating homogeneity of manufactured coated products.

With reference to FIG. 2, the flowchart illustrates one embodiment of the step of predefining a set of quality control (QC) inspection criteria that predicts or correlates one or more coated products having improved or consistent bioactive or biocompatible response 10. The step of predefining a set of quality control (QC) inspection criteria that predicts or correlates one or more coated products having improved bioactive or biocompatible response further comprises the steps of: providing or obtaining one or more coated products 20; characterizing the one or more coated products using one or more characterization methods to collect raw data 22; and generating one or more QC acceptance criteria that predicts or correlates the one or more coated products having improved and/or consistent bioactivity and/or biocompatible response 24. The predefining step 10 may further comprise preparing the one or more coated products for undergoing the one or more characterization methods (not shown).

In one embodiment, the step of providing, manufacturing or obtaining one or more coated products may comprise an article and at least one coating. The at least one coating is disposed on the article, the article includes a textile. The at least one coating is disposed on the outer diameter of the textile. The at least one coating is embedded or partially embedded through the textile.

In another embodiment, the step of providing, manufacturing or obtaining one or more coated products may comprise a product or an article, a first coating or coating layer and a second coating or coating layer. The product or article may comprise an outer surface, a product length, and a product material. The product material comprises a flexible biocompatible, non-biodegradable polymer or a rigid biocompatible, non-biodegradable polymer. The flexible biocompatible, non-biodegradable polymer may comprise a textile. The textile may include a suture or suture anchor. The rigid biocompatible, non-biodegradable polymer may comprise a rigid anchor. The first coating is disposed on the product or article. The first coating may comprise a biocompatible, non-biodegradable polymer. At least a portion of the second coating is disposed onto the first coating. At least a portion of the of the second coating is disposed onto the first coating and along the product length. The second coating comprises one or more bioceramic particles.

The textile may comprise a textile suitable for medical implantation purposes. The textile may include permanent high-strength orthopedic implants for repairing bone fractures or torn ligaments or tendons or vascular applications. Such textile implants may include flexible tissue anchors, cortical fixation devices like ACL loops, high-strength orthopedic sutures, bone cerclage cables, synthetic tendon and ligament grafts, interspinous spacers or spinal disc prostheses, spinal fusion devices, or synthetic scaffolds to repair bone voids. A flexible tissue anchor is a device for anchoring a suture to a bone and can be applied to attach or secure soft tissue to a bone, to attach or secure bone to bone, or to attach or secure bone to structures. Non-limiting examples of soft tissue include tendons, ligaments, fascia, skin, fibrous tissues, synovial membranes, fat, muscles, nerves, and blood vessels. Textile implants may further include vascular grafts.

The textile may comprise a textile construction, the textile construction includes a woven, a braided, a knitted construction, and/or any combination thereof. The textile may comprise a denier. The denier is a unit of measurement used to determine the fiber thickness of individual threads or filaments used in the creation of textiles and fabrics. The denier may comprise at least 200 denier or greater; the denier may comprise 300 denier or greater; the denier may comprise 400 denier or greater. The denier may further comprise 200 to 600 denier; it may comprise 400 to 600 denier. The textile may further comprise a picks-per-inch (PPI), the PPI may comprise at least 10 PPI or greater or 20 PPI or greater. The PPI may further comprise 10 to 40 PPI or 20 to 30 PPI.

The textile or product may comprise a material. The material may comprise a ceramic, polymer or metal. The polymer may further include a thermoset or thermoplastic polymer. The material may include a non-biodegradable material or polymer. The non-biodegradable polymers may comprise polyolefins, polyketones, polyamides, and polyesters. Suitable polyolefins include polyethylenes and polypropylenes, especially such polymers of high molar mass like ultra-high molar mass polyethylene (UHMWPE). Suitable polyamides include aliphatic, semi-aromatic and aromatic polyamides, like polyamide 6, polyamide 66 and their copolymers, and poly(phenylene terephthalamide). Suitable polyesters include aliphatic, semi-aromatic and aromatic polyesters, like poly(I-lactic acid) and its copolymers, polyethylene terephthalate (PET), polytrimethylene terephthalate (PTT), polyethylene naphthalate (PEN), polyethylene furanoate (PEF) and liquid crystalline aromatic copolyesters.

The material may further include biodegradable material. The biodegradable material may comprise a natural or synthetic biodegradable material. Natural biodegradable polymers include chitosan, silk fibroin, fibrinogen, collagen and hyaluronic acid. Synthetic biodegradable polymers include poly(ε-caprolactone) (PCL), PLA, PGA, copolymer PLGA, polytrimethylene carbonate (PTMC), and/or poly(p-dioxanone) (PDO). These materials have been proven to be biocompatible and have a controlled degradation rate, and their degradation products in-vivo have no toxic effects on tissues.

The material may further include synthetic, semi-synthetic polymers. Semi-synthetic or bio-derived biocompatible polymers include materials like derivates of proteins and polysaccharides, such as cellulose. Synthetic biocompatible polymers include materials like poly(meth)acrylates, polyolefins, vinyl polymers, fluoropolymers, polyesters, polyamides, polysulfones, polyacrylics, polyacetals, polyimides, polycarbonates, polyethylenes, polyurethanes, including copolymers, compounds and blends thereof. Such synthetic polymers may be based on natural compounds like amino acids and/or on synthetic monomers.

The product or textile may further comprise an outer surface, outer diameter or outer width. The width or outer diameter may comprise at least 1 mm or greater; at least 1.3 mm or greater; at least 2.2 mm or greater. The width or outer diameter may further comprise 1 to 5 mm; the width or outer diameter may further comprise 1 mm to 3 mm.

The at least one coating and/or a first coating may comprise a binder coating or binder coating layer. The binder coating may comprise a binder coating dispersion or solution that includes finely divided polymer particles and a non-solvent. The non-solvent is a liquid not able to dissolve or substantially dissolve the binder coating or binder coating layer. The binder coating or binder coating dispersion may further include an emulsifier or surfactant that is biocompatible. Preferably the non-solvent is an aqueous mixture or water. The person skilled in the art will be able to select suitable non-solvents and dispersion aids for a given coating polymer, or to select a commercially available dispersion that is suitable for use in present method based on present disclosures and his general knowledge. The polymer particles may comprise non-biodegradable and biocompatible polymer particles.

In another embodiment, the at least one coating, a first coating and/or binder coating solution or dispersion may comprise finely divided polymer particles and a solvent. The solvent may allow the polymer particles to be substantially or homogeneously dissolved. The person skilled in the art will be able to select a suitable solvent for a given binder coating based on his general knowledge, optionally supported by some experiments and/or literature. The solvent may comprise a tetrahydrofuran (THF), methyltetrahydrofuran (m-THF), dimethylformamide (DMF), dimethylacetamide (DMAc), dimethylsulfoxide (DMSO), dioxane, dioxolane, or mixtures thereof. Suitable non-solvents for use in the treating solvent include for example lower aliphatic alcohols like ethanol, aliphatic esters, aliphatic ethers, and lower alkanes and alkenes. As indicated above, the non-solvent can preferentially evaporate from a mixture forming the treating solvent during the method. The polymer particles may comprise non-biodegradable and biocompatible polymer particles.

In another embodiment, the at least one coating, a first coating and/or binder coating or binder coating dispersion may comprise finely divided polymer particles, a solvent and a non-solvent. The solvent and non-solvent may comprise miscible solvents. It was observed that a good solvent for the polymer may, in addition to swelling a surface layer, also solubilize the layer; which may result in partial removal of the binder coating, or in ceramic particles being completely enclosed or embedded by binder coating. It has been surprisingly found that varying the composition of such treating solvent mixture, provides the skilled person with a tool to influence the degree of embedding of the ceramic particles in the layer of binder coating on the textile. The polymer particles may comprise non-biodegradable and biocompatible polymer particles.

The finely divided polymer particles in a solvent and/or non-solvent comprise a concentration. The concentration of polymer may be chosen dependent on solubility and desired coating layer thickness. Generally, the concentration will be in the range 0.1-10 mass % of polymer particles in solvent. The solution contains e.g., at least 0.2, 0.5 or 1 mass %, and at most 8, 6, 4, 3 or 2 mass % of polymer particles.

The at least one coating, the first coating, and/or binder coating may be disposed onto a textile in different ways. The binder coating may be disposed onto an outer surface or outer diameter of the textile, product or article. The binder coating may be partially embedded within the textile or product. The binder coating may be fully embedded within the textile or product. Partially embedded means that the binder coating extends from the outer surface towards or through a portion of the wall thickness of the textile and/or product. Fully embedded means that the binder coating extends from the outer surface towards or through the wall thickness of the textile or product. The first coating or binder coating may be disposed onto the textile by dip coating or spray coating. After applying the solution or dispersion of the binder coating, the non-solvent or solvent is substantially removed. Removal may occur by evaporation or rinsing. The evaporation, if desired, may occur at elevated temperature to shorten time; to result in a layer of polymer particles on the textile. Furthermore, the solvent or non-solvent does not need to be completely removed at this stage, but a non-sticking or non-stick surface layer of the first coating or binder coating is preferred to prevent treated fibers of the textile from substantially adhering to each other.

The first coating or binder coating may comprise a binder coating thickness. In one embodiment, the thickness of the layer of the first coating or binder coating may be about half the size of the particles (taken as their d50 value, see hereinafter) of a second coating, the second coating including one or more bioceramic particles; so that the one or more bioceramic particles are partially embedded into the first coating and still can protrude from the first coating or binder coating layer. In one embodiment, the first coating or binder coating comprises at least 0.05 μm thickness. In further embodiments, the first coating or binder coating layer thickness comprises at least 0.01, 0.05, 0.1, 0.2, 0.3, 0.4, 0.6, 0.8, or 1 μm; whereas the layer thickness generally does not need to be more than 50, 40, 40, 20, 10, 5, or 2 μm. A relatively thin coating layer will have little effect on properties like flexibility of the textile and/or product.

The first coating or binder coating may further comprise a percent weight or mass increase (% w or % weight, % m or % mass) over the textile or product after the coating process. The thickness of the layer of the binder coating that is applied or the amount of binder coating applied may also be defined by the relative mass or weight increase of the coated product or coated textile after the coating process. The mass or weight increase upon coating the textile with binder coating is at least about 0.1, 0.2, 0.3, 0.4, or 0.5% mass, and/or at least 3, 2.5, or 2% mass.

The first coating or binder coating may further comprise a first coating material or a binder material. The binder material may comprise a ceramic, a polymer or a metal. The polymer may comprise a non-biodegradable polymer, a biodegradable polymer. The polymer may comprise a thermoset or thermoplastic polymer. The polymer may comprise a homopolymer, a copolymer and/or a block copolymer. The binder material may further include synthetic, semi-synthetic polymers. Semi-synthetic or bio-derived biocompatible polymers include materials like derivates of proteins and polysaccharides, such as cellulose. Synthetic biocompatible polymers include materials like poly(meth)acrylates, polyolefins, vinyl polymers, fluoropolymers, polyesters, polyamides, polysulfones, polyacrylics, polyacetals, polyimides, polycarbonates, polyethylenes, polyurethanes, including copolymers, compounds and blends thereof. Such synthetic polymers may be based on natural compounds like amino acids and/or on synthetic monomers.

In one exemplary embodiment, the first coating or binder material may comprise ultra-high molecular weight polyethylene (UHMWPE), lower density polyethylenes (LDPE), polyamide 66, polyurethanes; and/or polyethylene terephthalate (PET) with copolyesters. In other embodiments, the first coating or binder coating comprises a thermoplastic block copolymer. Block copolymers (or segmented) copolymers are polymers comprising blocks (also called segments) of polymers (including oligomers) that are chemically distinct, and that typically show different thermal and mechanical properties, and different solubilities. Often the blocks in a block copolymer comprising two (or more) types of blocks are referred to as being ‘hard’ and ‘soft’ polymer blocks, such different blocks resulting in microphase separation. The hard block in a block copolymer typically comprises a rigid or high modulus semi-crystalline or amorphous polymer, with—respectively—a melting temperature (Tm) or a glass transition temperature (Tg) higher than the use temperature, e.g. about 35° C. The soft block in the block copolymer often comprises a flexible, amorphous polymer with a Tg lower than 35° C., preferably lower than 0° C. Such thermoplastic block copolymers showing flexibility or elastomeric character are generally referred to as thermoplastic elastomers, or TPEs.

In another embodiment, the first coating material or binder material comprises a TPE material. The TPE comprises hard and/or soft blocks. The hard block comprises a polymer chosen from the group consisting of polyesters, polyamides, polystyrenes, polyacrylates, polyurethanes, polyolefins and/or any combination thereof. The soft block comprises a polymer chosen from the group consisting of polyethers, polyesters, polyacrylates, polyolefins and polysiloxanes, polyurethanes, and/or any combination thereof. Such polymers for the blocks are understood herein to include oligomers, homopolymers and copolymers, and polyesters are considered to include polycarbonates. Examples of TPE block copolymers are copolyester esters, copolyether esters, and copolycarbonate esters, wherein the hard blocks typically are based on semi-aromatic polyesters like polybutylene terephthalate (PBT); copolyester amides and copolyether amides; ethylene-propylene block copolymers; styrene-ethylene-butadiene block copolymers (SEBS); styrene-isobutylene block copolymers (SIBS); and polyurethanes comprising hard blocks based on diisocyanates and chain extenders, and polyester, polyether or polysiloxane soft blocks.

In another embodiment, the first coating material or binder material may comprise a TPE material, the TPE material comprises a polyurethane or a polyurethane block copolymer. The polyurethane TPE (also referred to as TPU) comprises as soft block an aliphatic polyester dial, an aliphatic polyether dial, or a polysiloxane dial. The hard blocks of a block copolymer for use in the method of the invention, including polyurethane TPE, may have a molar mass of about 160 to 10,000 Da, and more preferably about 200 to 2,000 Da. The molar mass of the soft segments may be typically about 200 to 100,000 Da, and preferably about 400 to 9000 Da. The ratio of soft to hard blocks can be chosen to result in certain stiffness or hardness of the polymer. Typically, durometer hardness of the polyurethane as measured with the Shore test using A or D scales (e.g., ShA or ShD), may be from 40 ShA, or at least 50 or 60 ShA and up to 80 or 75 ShD, generally representing a flexural modulus range of about 10 to 2000 MPa.

In another embodiment, the binder material comprises a TPE material, the TPE material comprises a polyurethane TPE, the polyurethane TPE comprises an aliphatic polyether or an aliphatic polyester as soft block. More specifically, the polyurethane TPE comprises an aliphatic polycarbonate. Suitable polyethers include poly(propylene oxide) dials, poly(tetramethylene oxide) dials, and their copolymers. Suitable aliphatic polyesters are generally made from at least one aliphatic dicarboxylic acid and at least one aliphatic dial, which components are preferably chosen such that an essentially amorphous oligomer or polymer is formed having a Tg below 10, 0, or −10° C. Aliphatic polycarbonate dials are based on similar aliphatic dials as used for other polyester dials, and can be synthesized via different routes as known in the art, such as a poly(hexamethylene carbonate) dial. Commercially available examples of such polymers include the Bionate® PCU products (DSM Biomedical BV). In one embodiment, the first coating or binder coating comprises Bionate®. Bionate® may comprise Bionate® PCU, Bionate® 55D, Bionate® 65D, Bionate® 75D, Bionate® 80A, Bionate® 90A, and/or any combination thereof.

In another embodiment, the first coating and/or binder coating may comprise a first coating or binder material and a second coating or binder material. In one embodiment, the first coating material or binder material is different than the second coating or binder material. In another embodiment, the first binder material is the same as the second binder material. Also, the first coating and/or binder coating may further comprise at least one additive. The at least one additive can be a targeted use of the coated product. The at least one additive may comprise antioxidants, processing aids, lubricants, surfactants, antistatic agents, colorants, radiopaque agents, fillers and/or any combination thereof. The at least one additive may be present in the typically effective amounts as known in the art, such as 0.01-5 mass % based on the amount of the polymer, preferably 0.01-1 mass %. In another embodiment, the binder coating does not include at least one additive.

The second coating may comprise a bioceramic coating or a bioactive ceramic coating or dispersion. The bioceramic coating comprises one or more bioactive ceramic particles and a solvent. Alternatively, the bioceramic coating may comprise one or more bioceramic particles and a non-solvent. In another embodiment, the bioceramic coating comprises a dispersion, the dispersion includes one or more bioceramic particles, a solvent or a non-solvent. The bioactive ceramic particles comprise all inorganic materials that show the capability of direct bonding to living bone, for example by formation of biologically active bone-like apatite through chemical reaction of the particle surface with surrounding body fluid resulting in a bactericidal action against microorganisms. The bioactive ceramic particles may also include Bioglass®, calcium phosphates, bioactive glass or bioglass or blends. Calcium phosphates may comprise dicalcium phosphate anhydrate (CaHPO₄; or DCPA), dicalcium phosphate dihydrate (CaHPO₄·2H₂O; DCPD), octacalcium phosphate (Ca₈(HPO₄)₂·5H₂O; or OCP), tricalcium phosphate (Ca₃(PO₄)₂; or TCP), and hydroxyapatite (Ca₁₀(PO₄MOH)₂; or HA).

In one embodiment, the bioceramic coating or coating layer may comprise bioactive glass or BioGlass®. Bioglass® refers to mixed organic oxides that have a surface-reactive glass film compatible with tissues; and may be used as a surface coating in some types of medical and dental implants. The bioactive glass or Bioglass® comprises molecules, elements or ions, including Sodium (Na), Phosphorus (P), Silicon (Si), and Calcium (Ca). It is highly desired or desirable that at least a portion of one or more of these elements are partially embedded or a portion sits below the first coating or binder coating to allow exposure of the elements to the bodily fluids after implantation. It is imperative that the elements are exposed to the bodily fluids to facilitate the sequence of chemical reactions to stimulate osteogenesis and antimicrobial activity and/or any other bioactive response.

Immediately after the implantation of bioactive glass, the exposure of the bioceramic particles to body fluids causes the exchange of network-modifier ions (e.g., Na⁺, K⁺, Ca²⁺) with H⁺ or H₃O⁺ ions from surrounding body fluids. This reaction gives rise to an increase in the local pH (from 7 to 10), resulting in an alkaline microenvironment. Additionally, the release of sodium, silica, calcium, and phosphate ions from the bioactive glass surface enhances the salt concentration and the osmotic pressure. These two mechanisms of action efficiently inhibit bacterial growth and, consequently, the adhesion and contamination of implants. The release of protons within the surroundings causes the hydrolysis of the silica groups and, therefore, the formation of silanol (Si—OH) surface groups and the development of a silica gel-based layer. Because of the exchange of alkali ions, the silica gel-based layer (SiO₂-based layer) increases in thickness and gathers calcium and phosphate ions (CaO—P₂O₅) already present in the body fluids, creating a stratum rich in amorphous calcium phosphate phase on top of the SiO₂-based layer. The amorphous CaO—P₂O₅ phase crystallizes as it bonds to hydroxide and carbonate anions from the surrounding environment, and gradually transforms into a hydroxyapatite (HA) layer over the surface of implanted bioactive glass particles, starting the process of osseointegration. The HCA crystals bind to collagen fibrils formed by local osteoblasts, generating a strong bond to bone and/or tissue.

In one embodiment, the one or more bioceramic particles may comprise Bioglass®. The Bioglass® may comprise Bioglass® 45S5 grade, which is indicated to be a glass comprised of the elements of 45 mass % Silicon Dioxide (SiO₂), 24.5 mass % Calcium Oxide (CaO), 24.5 mass % Sodium Oxide (Na₂O), and 6.0 mass % Phosphorous Pentoxide (P₂O). The high ratio of calcium to phosphorus in this material would promote formation of HA crystals; calcium and silica ions can act as crystallization nuclei.

In another embodiment, the bioceramic coating may comprise a first one or more bioactive ceramic particles, a second one or more bioactive ceramic particles and a treating solvent. The treating solvent may comprise a standard solvent or a non-solvent. The treating solvent may comprise a solvent and a non-solvent. Alternatively, the bioceramic coating may comprise a first one or more bioactive ceramic particles and a second one or more bioactive ceramic particles within the dispersion. The first one or more bioactive ceramic particles may be different or the same as the second one or more bioactive ceramic particles. The first one or more bioactive ceramic particles may comprise Bioglass® and a second one or more bioactive ceramic particles may comprise HAp. The second one or more bioactive ceramic particles may comprise small or trace amounts of other (inorganic) elements or ions, like Na, Mg, Fe, Zn, Ti, Ag, Cu or SO₄, or CO₃ which may improve specific properties of the bioactive ceramic particles.

The one or more bioactive ceramic particles may comprise a particle size. The particle size comprises a range of 0.01-10 μm. In another embodiment, the bioactive ceramic particle size may comprise a size smaller than 10 μm. In other embodiments, the ceramic particles size includes size of at least 20, 30, 50, 100, 200, 300, 400, or 500 μm. In other embodiments, the ceramic particle size may comprise at least 20 nm or greater. Further embodiments, the ceramic particles size comprises a size of at least 8, 7, 6, 5, 4, 3, 2 μm, or at most 1 μm. The particle size and particle size distribution can be measured with SEM or optical microscopy, or with (laser) light diffraction techniques. For example, a d50 value was measured with light diffraction according to ISO 13320:2009, e.g., with a Malvern Mastersizer 2000 to help define the particle size of the bioceramic particles. Changing the particle size from μm to nm sizes and/or larger to smaller may increase surface area, allowing the release of more alkaline species, and consequently may display an enhanced microbial effect. In one embodiment, the first one or more bioceramic particles comprises a first bioceramic particle size and the second one or more bioceramic particles may comprise a second bioceramic particle size. The first bioceramic particle size may be the same or different than the second bioceramic particle size.

The second coating and/or bioceramic coating may comprise a percent mass (% mass) of bioactive ceramic particles within a treating solvent. The treating solvent may comprise a solvent or non-solvent. The % mass of bioactive ceramic particles in a treating solvent comprises 1-25% mass. In another embodiment, the % mass of bioactive ceramic particles in treating solvent or non-solvent comprises at least 22, 20, 18, 16, 14, 12 or 10% mass. In another embodiment, the % mass of bioactive ceramic particles in treating solvent or non-solvent comprises at least 2, 3, or 5% mass of bioactive ceramic particles.

The solvent or non-solvent used within the second coating and/or the bioceramic coating may comprise a treating solvent that interacts with the first coating and/or binder coating. The interaction of the solvent or non-solvent with the binder coating may include short contacting times to allow the solvent to effectively modify the surface of the binder coating, but evaporated or removed prior to implantation of the coated product. Modification of the surface of the binder coating may include swelling of a surface layer of the first coating and/or binder coating to tackify the surface layer of the binder coating, and/or solubilize the surface layer of the binder coating. The solubilization may result in partial removal of the binder coating to allow the second coating and/or the bioceramic coating to be deposited on the first coating and/or binder coating or to be embedded or partially embedded into the first coating and/or binder coating or textile. The swelling of the surface layer may allow at least a portion of the one or more bioceramic particles sink or partially sink below the first coating and/or binder coating layer, resulting in a partially exposed one or more bioceramic particles. This may allow the second coating bioceramic coating to be deposited on the binder coating surface, or to be embedded into a portion of the binder coating or fully embedded into a portion of the binder coating. In another embodiment, the second coating and/or the bioceramic coating may comprise one or more bioceramic particles and a non-solvent.

The solvent may comprise a tetrahydrofuran (THF), methyltetrahydrofuran (m-THF), dimethylformamide (DMF), dimethylacetamide (DMAc), dimethylsulfoxide (DMSO), dioxane, dioxolane, or mixtures thereof. Suitable non-solvents for use in the treating solvent include for example lower aliphatic alcohols like ethanol, aliphatic esters, aliphatic ethers, and lower alkanes and alkenes. As indicated above, the non-solvent can preferentially evaporate from a mixture forming the treating solvent during the method.

In another embodiment, the second coating, the bioceramic coating or the bioceramic coating dispersion may comprise a solvent and a non-solvent (e.g., or a treating solvent). The solvent and non-solvent may comprise miscible solvents. It was observed that a good solvent for the first coating or binder coating may, in addition to swelling a surface layer, also solubilize the first coating or binder coating, which may result in partial removal of the first coating binder coating, which allows the one or more ceramic particles being partially embedded. It has been surprisingly found that varying the composition of such treating solvent mixture, provides the skilled person with a tool to influence the degree of embedding of the one or more bioceramic particles in the layer of first coating or binder coating.

The solvent and/or a non-solvent for the second coating or dispersion may be present in a percent volume (% Vol) within the bioceramic coating or the bioceramic coating dispersion. In one embodiment, the % volume may comprise a range of 2% to 98% volume. In another embodiment, the % volume may comprise at least 90, 80, 70, 70, 60, 50, 40, 30, 20, 10, 5 or 2 vol %.

The deposition or depositing of the second coating, the bioceramic coating and/or bioceramic coating dispersion may comprise dip coating and spray coating onto the first coating or binder coating. Such coating methods allow to apply a thin layer of the dispersion on the surface of a complex shaped article like a textile within short time, optionally using multiple coating steps with intermediate drying, and with controllable contact time of coating polymer and dispersion, before removing excess dispersion and/or removing at least part of the treating solvent, e.g., by drying/evaporating and/or by rinsing with a rinsing solvent. Treating can be suitable performed at ambient conditions, but for example the temperature may also be increased, e.g., to shorten contacting and subsequent drying times.

In one embodiment, the second coating or bioceramic coating is deposited onto the first coating or binder coating by dip coating. The dip coating may comprise submerge time, the submerge time is the total time the coated product, coated textile or textile is submerged in the bioceramic coating or the bioceramic coating dispersion. The submerge time includes periods of 1-20 seconds. Alternatively, the bioceramic coating is deposited onto the binder coating may a plurality of coating steps. By applying multiple short dip coating steps, the surface coverage can be more controlled with ceramic particles, rather than aiming to obtain a certain coverage in one step. The plurality of dip coating steps may include at least 2, 3, 4, 5, 6, 7, 8, 9 or 10 dip coating steps, optionally using intermediate drying periods to remove at least part of the treating solvent. A drying period can vary from 1 to 10 min, depending on conditions and volatility of treating solvent (or solvents contained therein). Suitable temperatures for coating and drying are in the range 10 to 150° C., depending on the softening temperature of the fibers of the textile or the binder coating; and is typically about 40-60° C., optionally in combination with reduced pressure and/or inert gas, like nitrogen flow. In another embodiment, the bioceramic coating is deposited onto the binder coating by spray coating. Spray coating allows the application of a plurality of thin layers after each attempt with preferably intermediate drying, for similar reasons as mentioned above for dip coating.

The solvent or non-solvent may be removed or substantially removed by a variety of different techniques. These techniques include evaporation, drying or rinsing. Drying conditions are dependent on volatilities of components to be removed, and the skilled person can determine suitable conditions. Drying can be done in ambient conditions, but also at elevated temperatures, under reduced pressure and/or by applying an inert gas flow. Rinsing aims to completely remove residual solvent or non-solvent and possibly other unwanted compounds, to make an article that will comply with requirements for medical implants.

In another embodiment, the bioceramic coating may comprise a total increase of percent weight (% wt) or percent mass (% mass) after coating application. The amount of bioceramic particles deposited onto the binder coating may be defined by the relative mass increase of the coated textile or product. The % weight or % mass increase of the coated fibrous product with a binder coating after application of the bioceramic coating or bioceramic dispersion and after removing or substantially removing the treating solvent or non-solvent comprises at least a 0.1, 0.2, 0.3, 0.4, or 0.5% mass increase. In another embodiment, the % mass increase is at least 20, 17, 15, 12, 10, 7, 5, 4, 3, 2.5, or 2% mass increase.

In another embodiment, the at least one coating, a first coating and/or a second coating may further comprise at least one metal oxide. The at least one metal oxide comprises iron, manganese, zinc, cobalt, gold, silver, copper and/or any combination thereof. Metal ions can enhance the bioactive responses of bioactive glass. More specifically, it can enhance the antimicrobial effect. Metal ions contain broad spectrum activities and interact with many different microbial intracellular components, resulting in the disruption of vital cell functions and eventually cell death.

The method of quality controlling the manufacturing process may comprise the step of selecting a sample or subset of one or more coated products. The step of selecting one or more coated products may comprise nominal and worst-case (e.g., outer limits) test runs to produce a coated textile product that are representative of routine processing (nominal test runs) and/or processing at the outer limits of acceptance criteria (worst-case test runs). In one embodiment, a sample or subset, N, of the one or more coated products may comprise greater than or equal to 1 sample. In another embodiment, a sample or subset of the one or more coated products may comprise at least 30 samples. Alternatively, a sample or subset of the one or more coated products may comprise a minimum number of samples to achieve a 90% confidence interval to infer information on the entirety of the one or more coated product manufacturing lots. In another embodiment, the entirety of the one or more coated product manufacturing lots may be sampled and tested.

Relevant statistical methods known in the art may be used to select the proper sample size required for characterization testing. In one embodiment, the Clopper-Pearson (exact) statistical method is selected and reveals that a sample size of n=30 may be sufficient to establish QC acceptance criteria that can repeatably predict optimal healing responses in the body for the one or more coated product at a 90% or 95% confidence. For example, when considering an experiment with 30 samples (n=30) and with 30 successes (“pass”), the Clopper-Pearson (exact) method gives the following 95% and 90% confidence intervals for a binomial probability: 95% confidence interval 0.88430≤p≤1.00000, 90% confidence interval 0.90497≤p≤1.00000. Therefore, when 30 of 30 samples meet requirements, it can be stated with 95% certainty that the probability of any subsequent experiment passing is greater than or equal to 88.430%, and with 90% certainty the probability of any subsequent experiment of passing is greater than or equal to 90.497%. Furthermore, the collected raw data or the sample data of the one or more coated products samples may be normalized to a relative standard deviation (% RSD). % RSD is a method used to determine the precision of the average results obtained. The % RSD is determined by taking the standard deviation of a set of raw or sample data, multiplying it by 100, and dividing the result by the average of the same measurements.

In another embodiment, the “Shapiro-Wilk” statistical method may be used to estimate sample size and test its normality for smaller sample sizes. Once normality is confirmed, the statistical tolerance limits are calculated using the inverse cumulative distribution function for the non-central t distribution, as specified by NIST (7.2.6.3. Tolerance intervals for a normal distribution (nist.gov)). These calculations allow for the determination of the QC criteria minimum coating wt. % (within a 90% confidence interval) that ≥90% of applicable samples would pass the desired QC criteria.

As disclosed herein, the method for quality control inspection of coated products comprises characterizing the one or more coated products using one or more characterization methods to collect raw data and/or sample data 14, 22. The step of characterizing the one or more coated products using the one or more characterization methods may further comprise the step of: preparing the one or more coated products to undergo the one or more characterization methods.

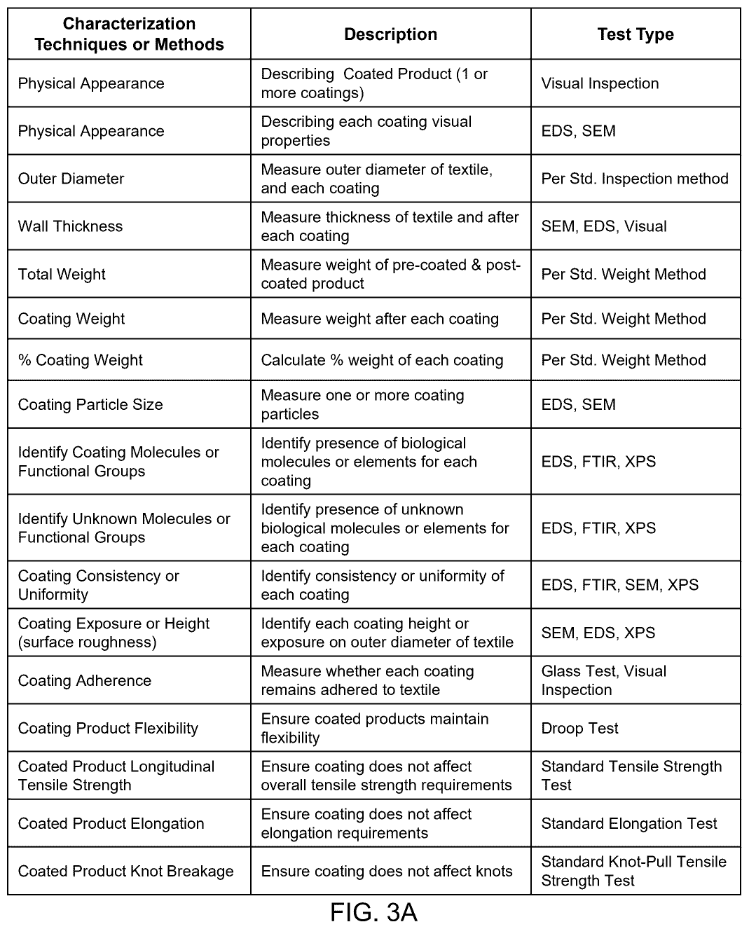

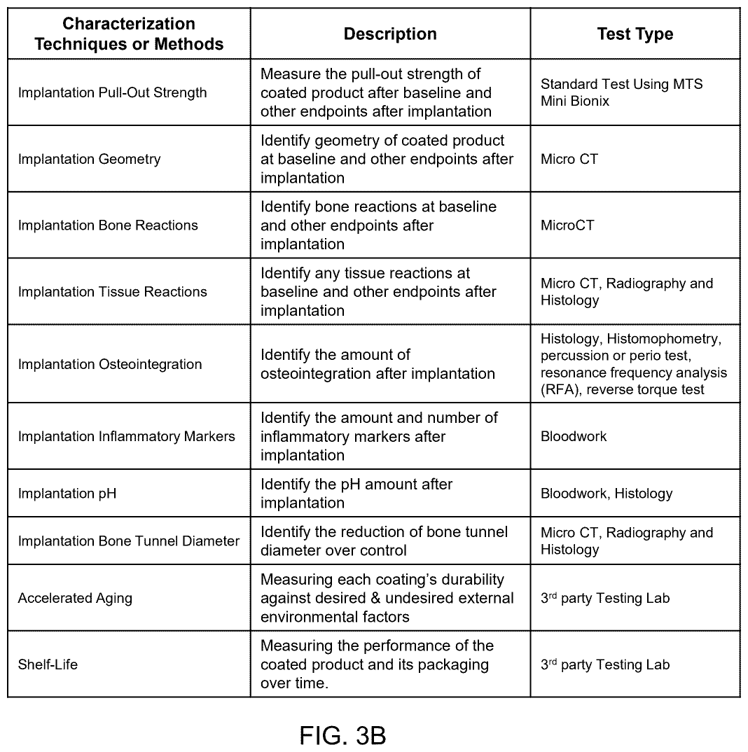

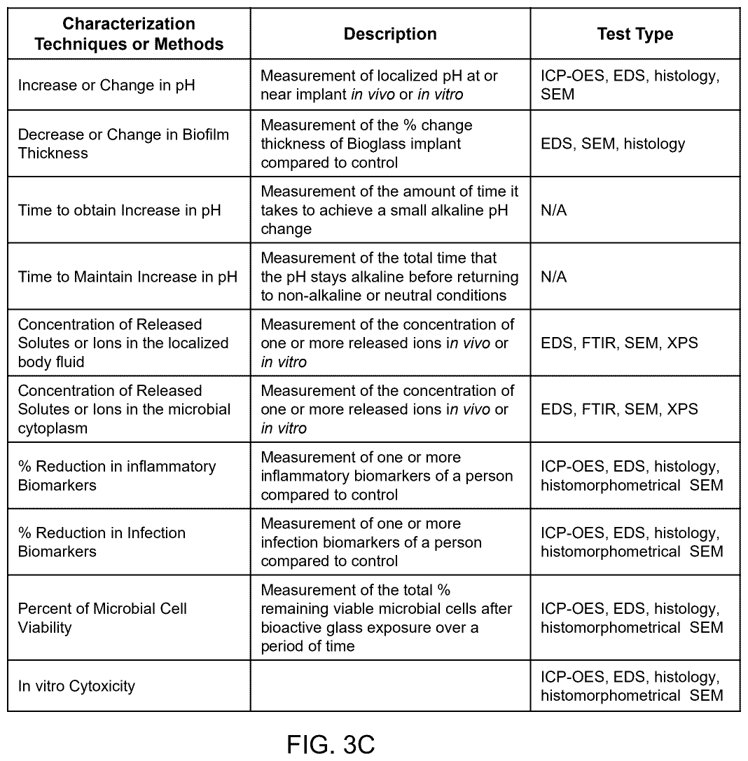

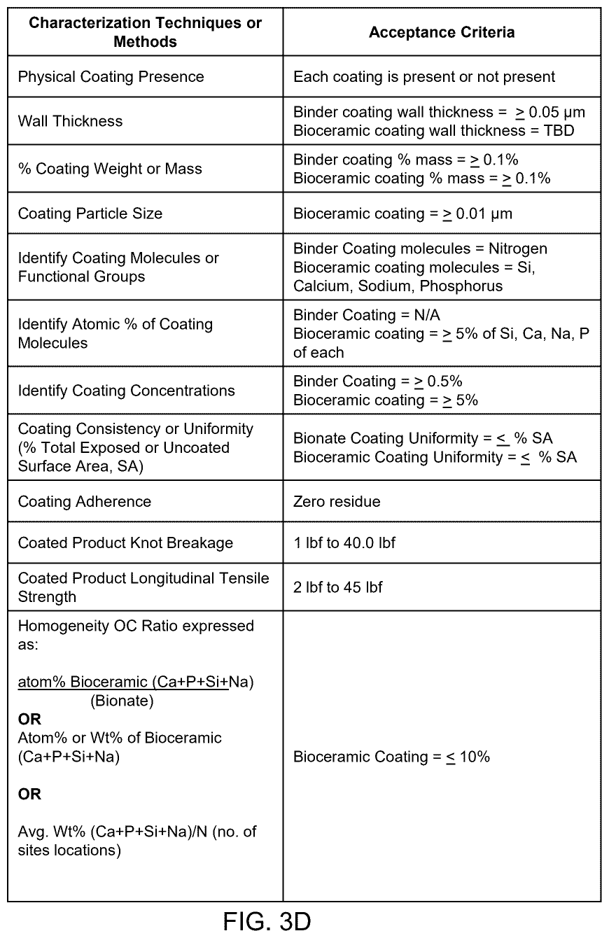

With reference to FIGS. 3A-3D, the tables disclose the one or more available characterization methods to help determine that the selected coating parameters or coating manufacturing parameters can help predict or substantially predict that the one or more coated products can provide for a reproducible and repeatable coatings to enable a consistent or optimized healing response after implantation. More specifically, the step of characterization of the one or more coated products is important to accurately predict or correlate the second coating containing the enhanced bioactive and/or biocompatible properties (e.g., healing response) of the one or more coated products is homogeneously applied and its successful clinical outcome.

Such control of the chemical, physical, and mechanical properties or coating parameters of the at least one coating, a first coating and/or a second coating parameters and its deposition process may help manufacture a reproducible coated product that can consistently and effectively predict or substantially predict the biological properties or healing response in a body. The healing response or healing response properties in-vivo may be achieved by reducing an inflammatory foreign body reaction at the local implantation site or uniformly throughout the body, reducing the degradation rate of the coated product to reduce any effect on the tissue repair, enhancing osteointegration at the implant site, increasing or improving cell adhesion, proliferation and/or cell-to-cell interactions, decreasing bone tunnel diameter, increasing pull-out strength, and increasing longevity of the implant within the implant site.

The one or more characterization methods may be performed on a pre-coated textile, a coated textile after a first coating, a coated textile after a second coating, a coated product after in-vitro testing, a coated product after implantation, and/or any combination thereof. The one or more characterization methods may include data at one or more desired or set locations along the length of a sample of a textile or coated product, data at one or more set locations around the circumference of the sample of a textile or coated product, and or data at one or more set locations around the circumference and along the length of the sample of a textile or coated product. Each of the one or more set locations may be spaced apart at a distance. In one embodiment, the one or more characterization methods may include data from at least one desired location along the product length of the one or more coated product. Alternatively, the one or more characterization methods may include data from a plurality of locations along the product length of the one or more coated product. The plurality of locations may include at least 3 or 4 locations.

The one or more characterization tests may comprise physical property characterization tests. The physical property characterization tests may include physical appearance, outer diameter, wall thickness of each coating, % weight or mass increase of each coating, particle size of each coating, consistency or uniformity of each coating, exposure or height of each coating (e.g., surface roughness), coating deposition (e.g., whether each coating is on the outer surface of the textile, partially embedded or fully embedded), and/or any combination thereof. These physical properties may be performed by a variety of different analytical techniques, including visual inspection, microscope, scanning electron microscope (SEM), energy dispersive X-Ray spectroscopy (EDS), optical microscopy (OM), ion scattering spectroscopy (ISS), electron spectroscopy for chemical analysis (ESCA), also called X-ray photoelectron microscopy (XPS), secondary ion mass spectroscopy (SIMS). To analyze the samples or a subset of the one or more coated products, each analytical technique may require a desired set-up to be established, including a working distance (WD), a desired beam energy (KeV), a desired magnification (x), a desired resolution, a desired sample surface area, desired or discrete measurement locations on the on each sample of the one or more coated products. Furthermore, prior to the analysis of each one or more samples of the one or more coated products may require initial preparation.

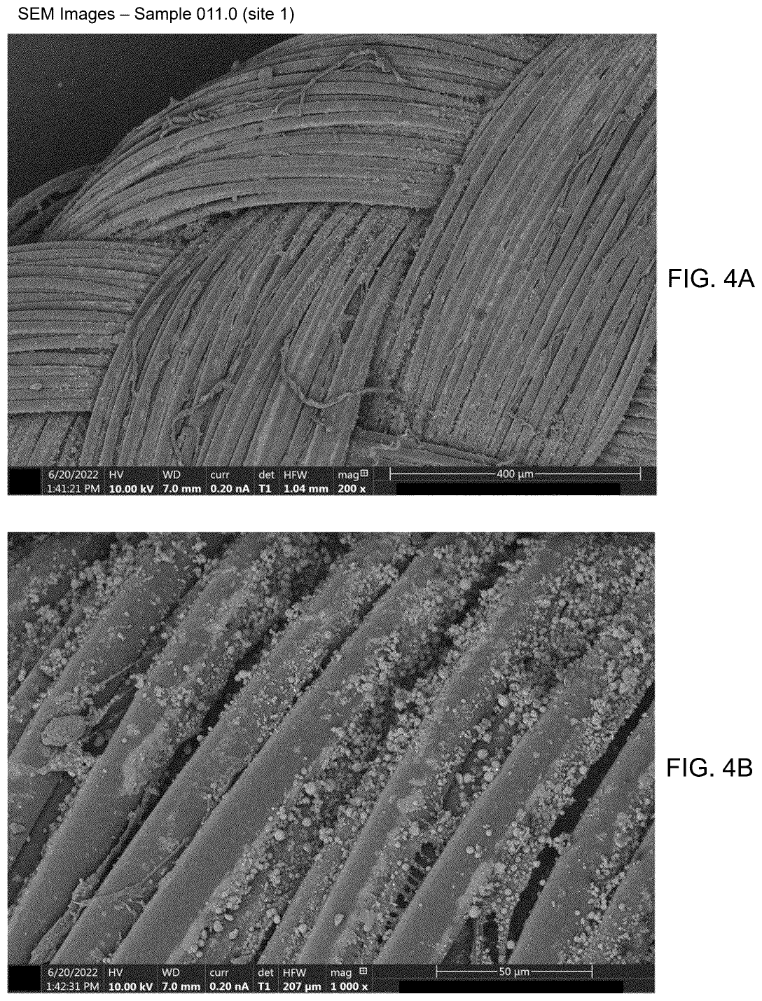

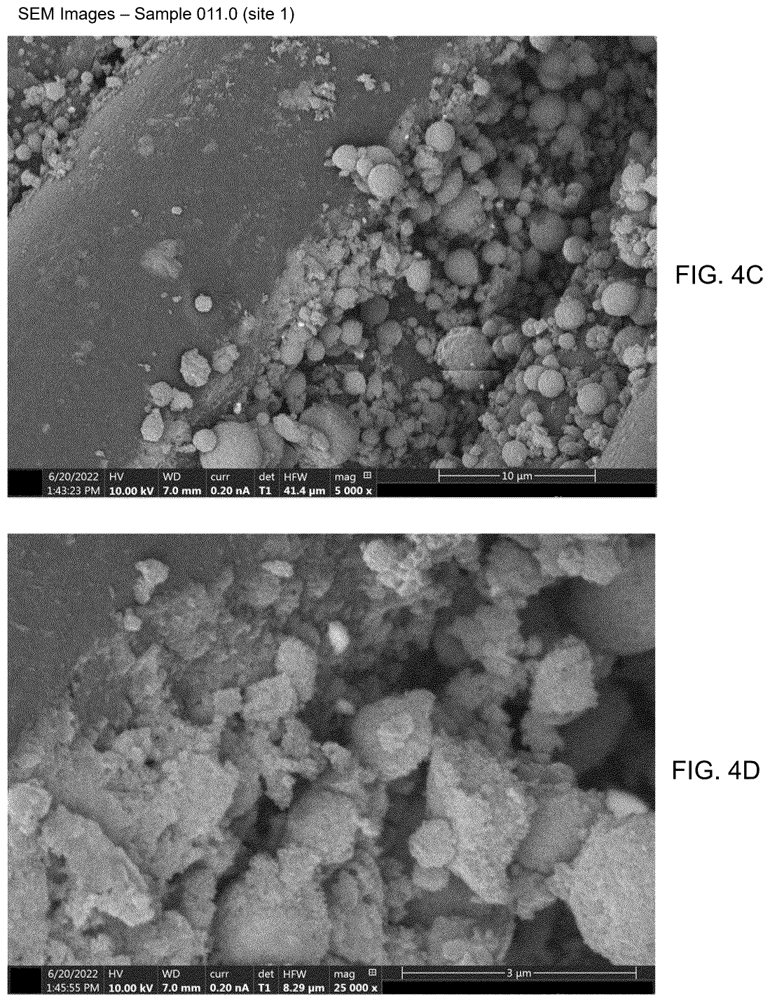

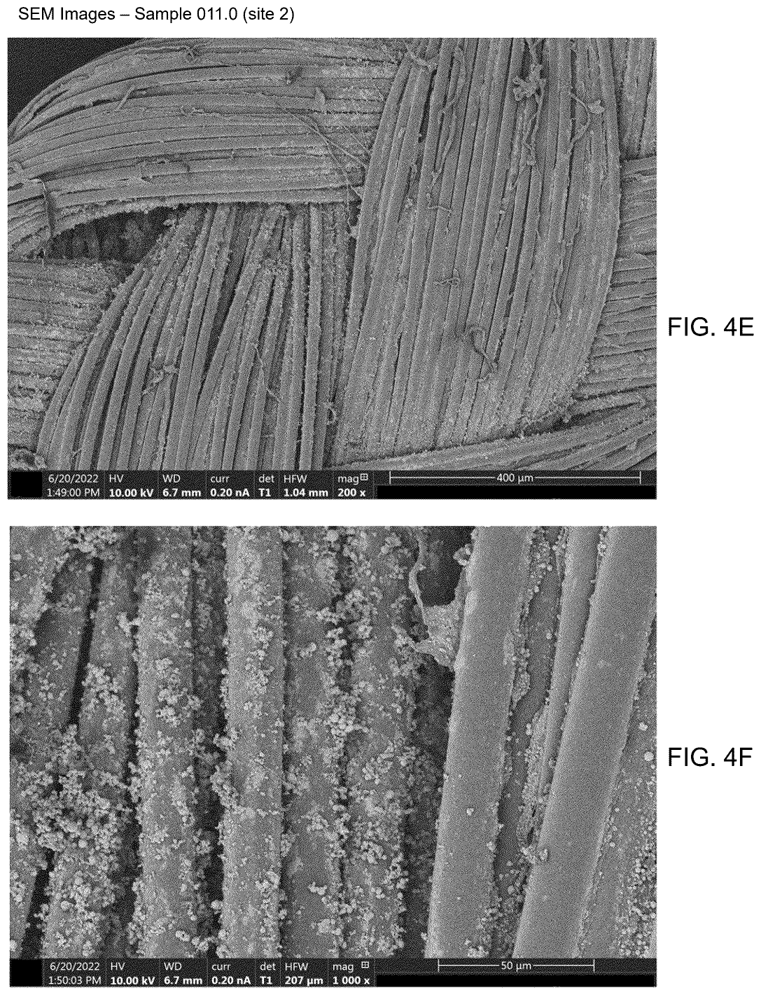

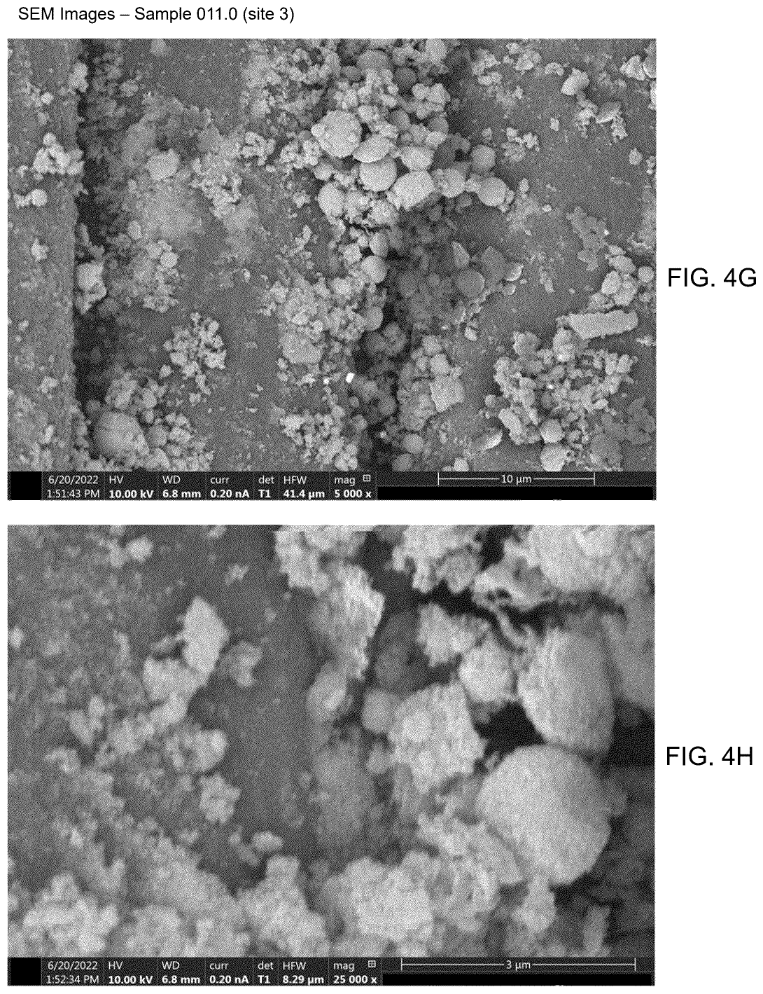

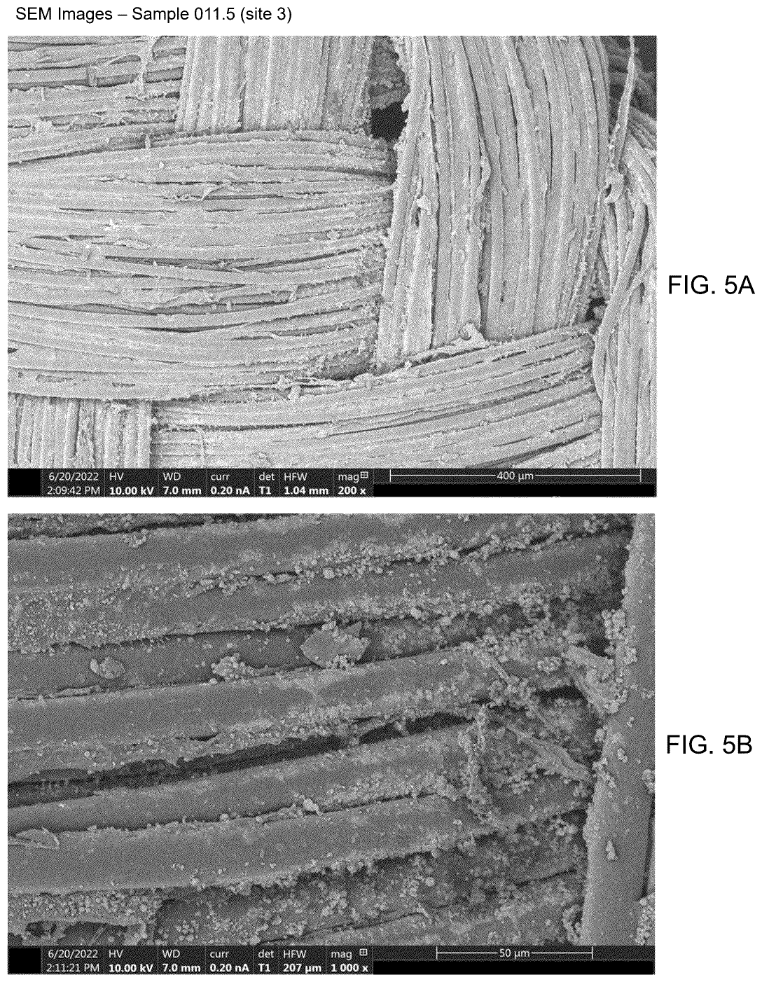

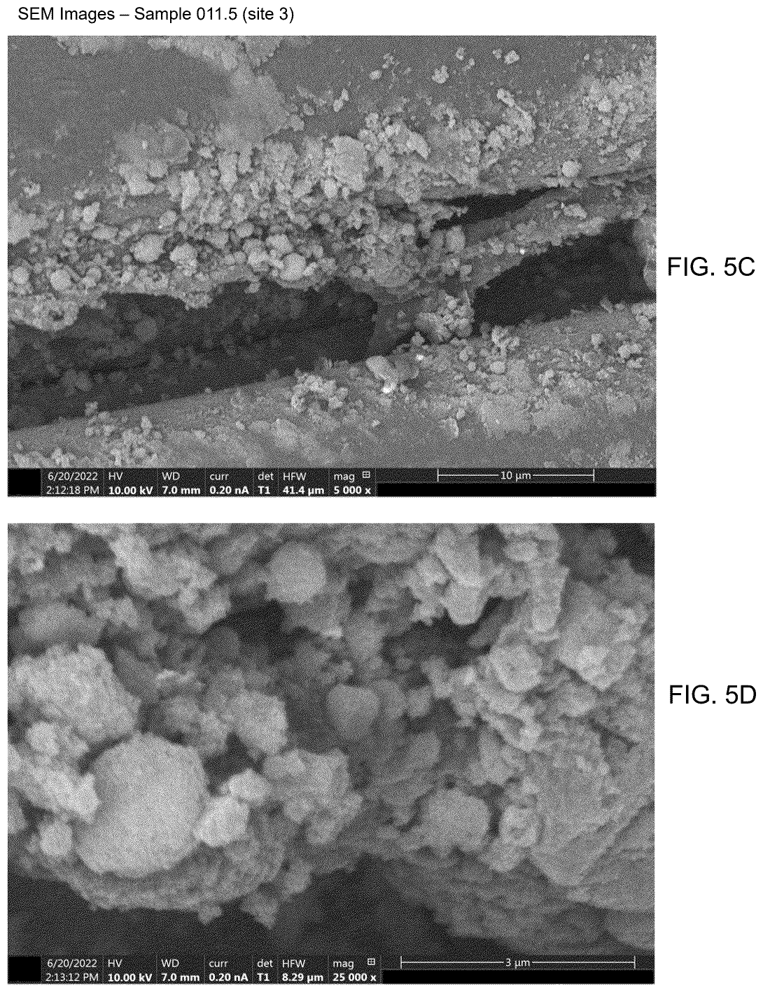

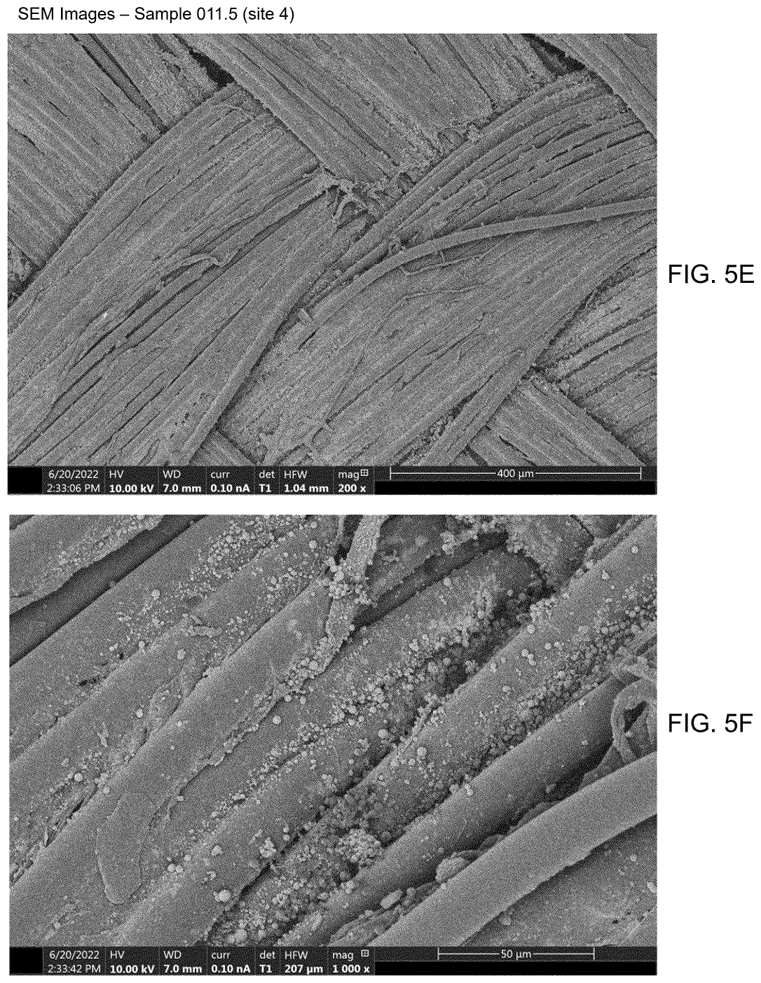

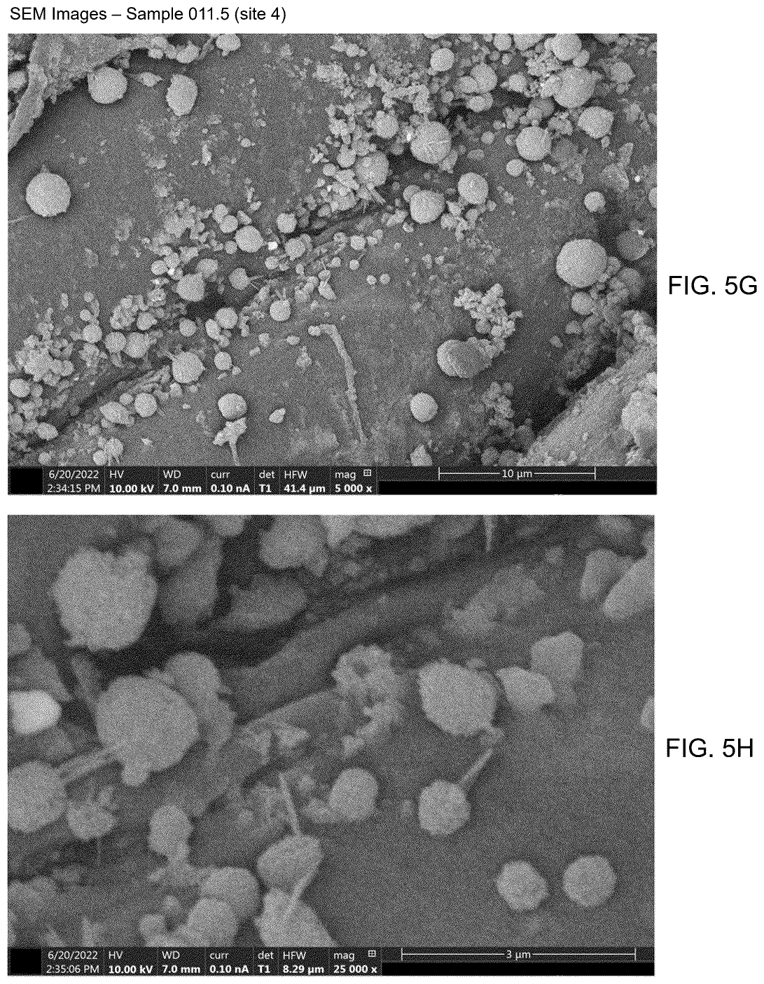

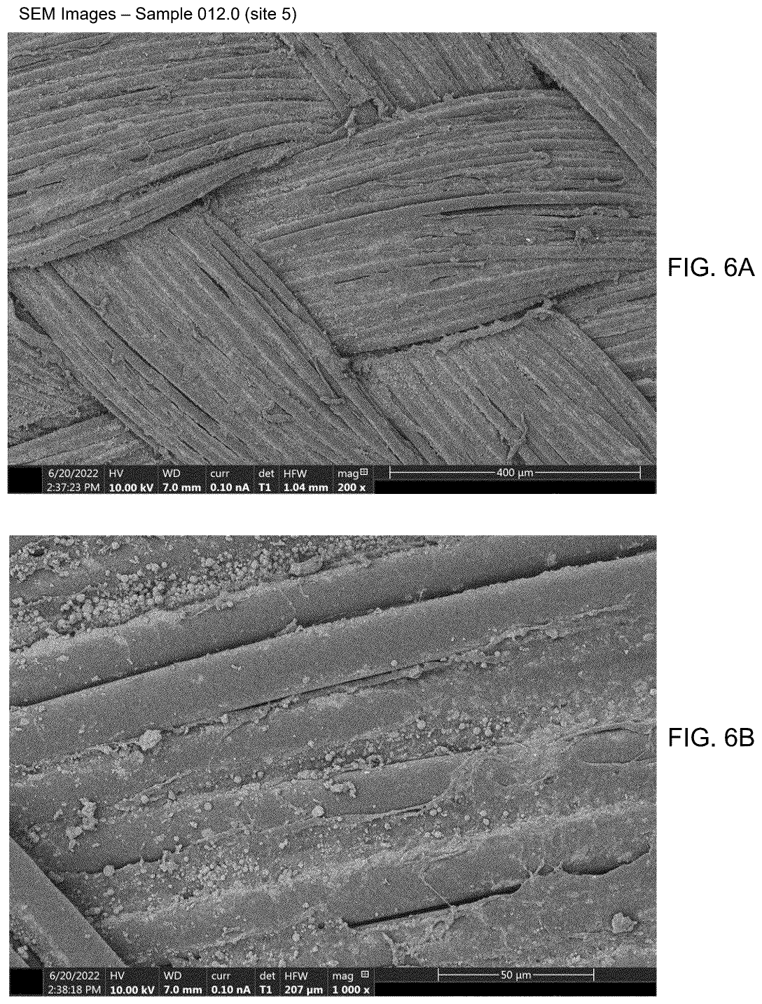

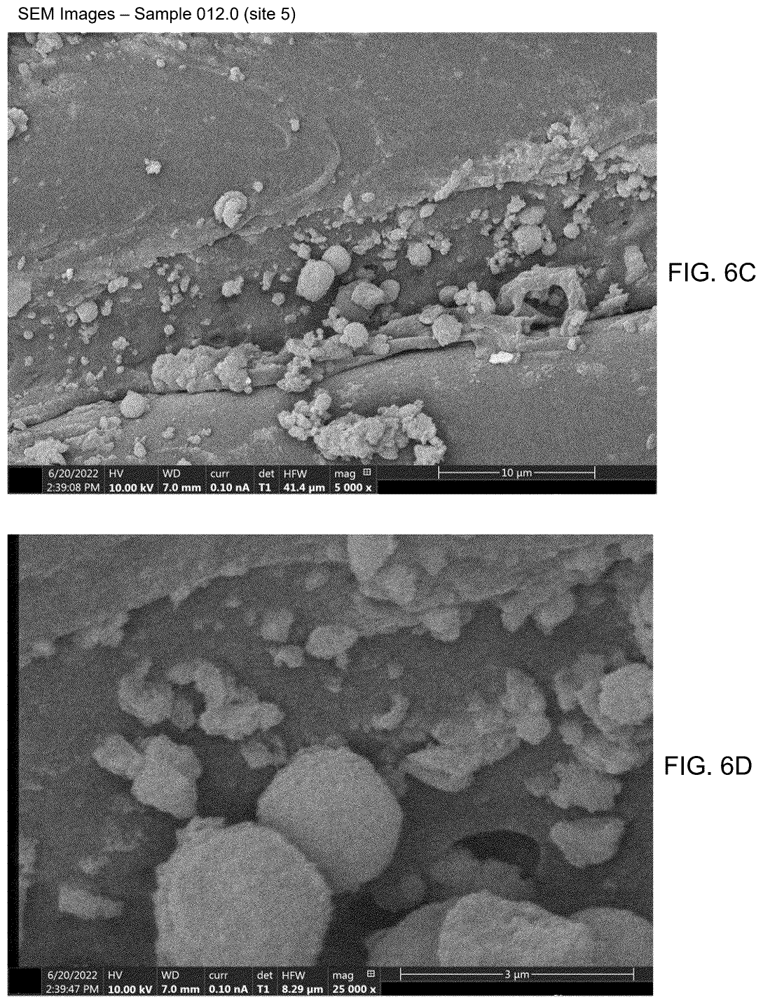

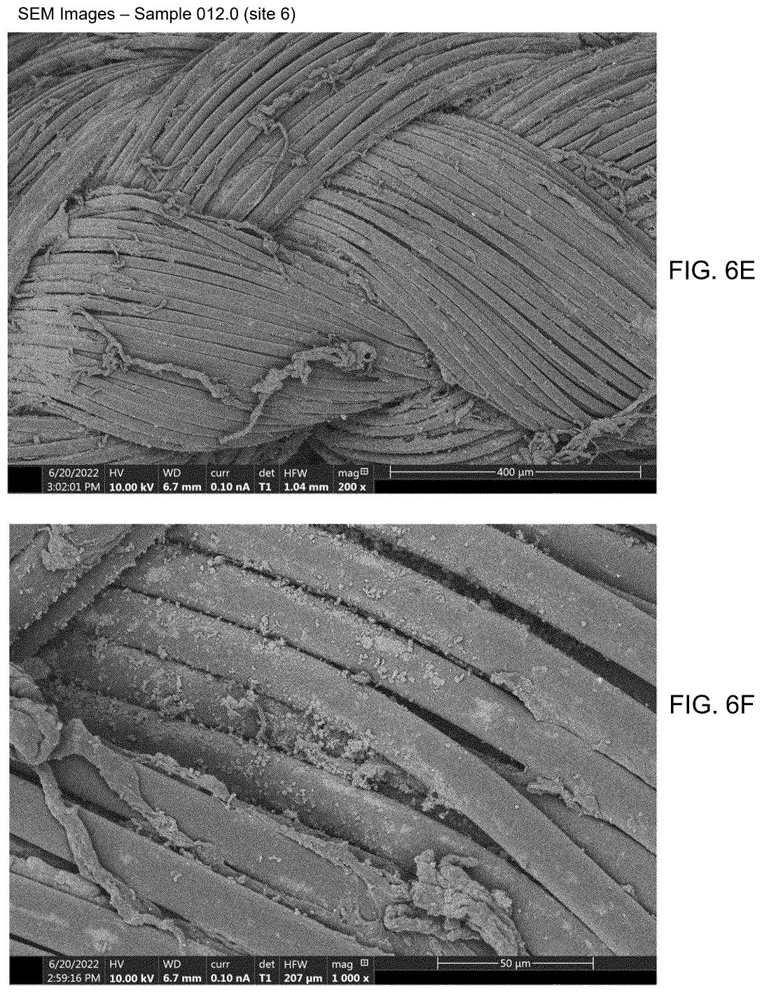

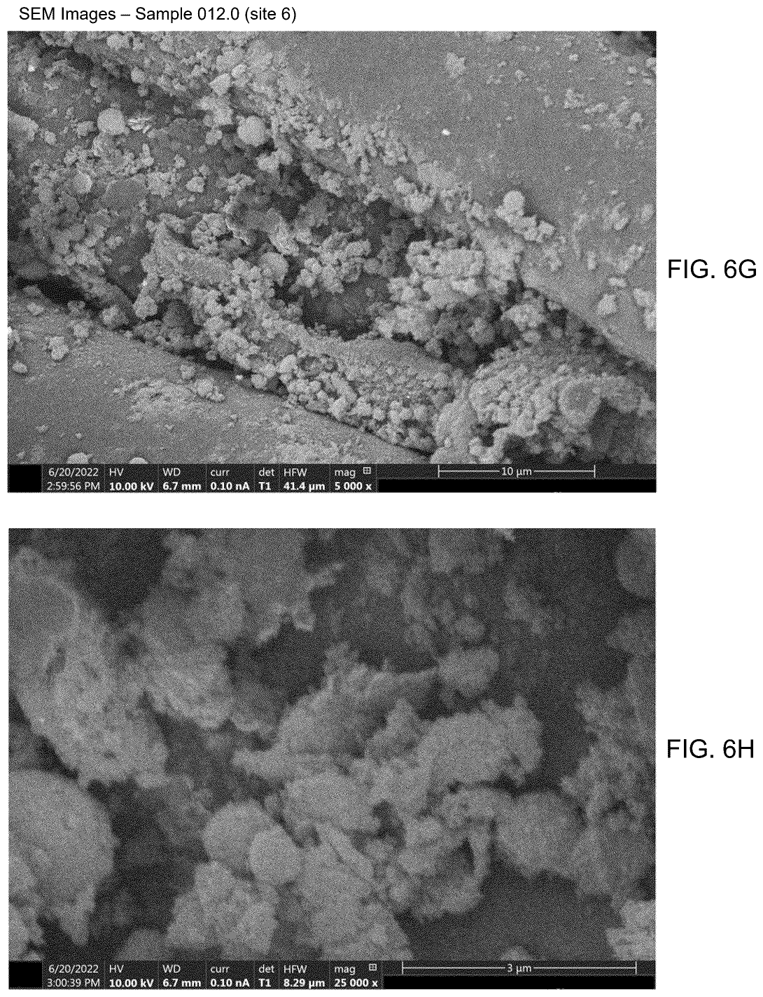

With reference to FIGS. 4A-4H (Sample 011.0, Sites 1-3), 5A-5H (Sample 011.5, Sites 3-4), 6A-6H (Sample 012, Sites 5-6) illustrate SEM images of one or more coated products surface characteristics. The SEM images were taken at different magnifications of 200× to 25,000×, at a working distance of 6 mm to 8 mm, and/or voltages of 5 kv to 15kv for one or more coated textile products, Sample 011.0, 011.5, and 012. Several SEM images were taken at a plurality of site locations along the coated textile product for visual inspection and physical characterization. The colored boxes indicate the desired site location on the one or more coated products. The SEM images revealed measurements of the one or more bioceramic particles sizes. The bioceramic particles resulted in sizes of less than 3 μm. The SEM images illustrate that the coated product contains particle agglomerates, mostly congregating on the sides of the coated textile product and where the braids overlap. Furthermore, the SEM images show the exposed bioceramic particles disposed over the first coating.

The one or more characterization tests may comprise chemical properties tests performed by one or more analytical techniques. The chemical properties tests may include the identification of the textile and/or each coating composition, identification of one or more elements or molecules of each of the textile and/or each of the coatings within each composition, identification of the amount of the one or more elements of each of the textile and/or each of the coatings (e.g., to be expressed as % weight, % mass % volume or % atomic percent), and identification of unwanted or undesired compositions and/or molecules within each compositions, and/or any combination thereof.

The chemical properties tests may be performed by a variety of different analytical techniques, including visual inspection, auger electron spectroscopy (AES), energy dispersive X-Ray spectroscopy (EDS), ion scattering spectroscopy (ISS), electron spectroscopy for chemical analysis (ESCA), also called X-ray photoelectron microscopy (XPS), secondary ion mass spectroscopy (SIMS), x-ray photo electron spectroscopy (XRD), x-ray diffraction (XRF), Fourier transform infrared (FTIR), and/or any combination thereof. To analyze the samples or a subset of the one or more coated products, each analytical technique may require a desired set-up to be established, including a working distance (WD), a desired beam energy (KeV), a desired magnification (x), a desired resolution, a desired sample surface area, desired or discrete measurement locations on the on each sample of the one or more coated products. Furthermore, prior to the analysis of each one or more samples of the one or more coated products may require initial preparation.

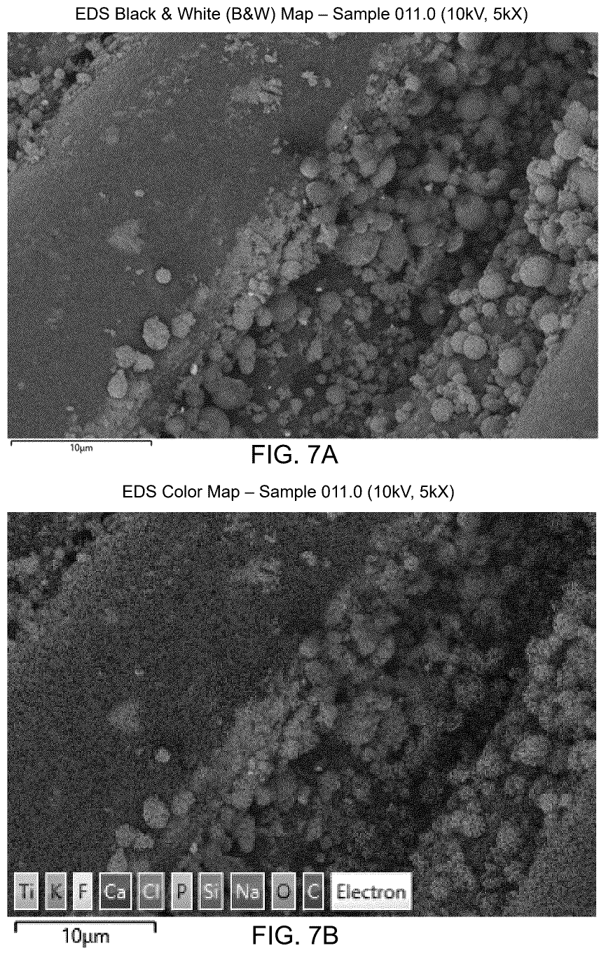

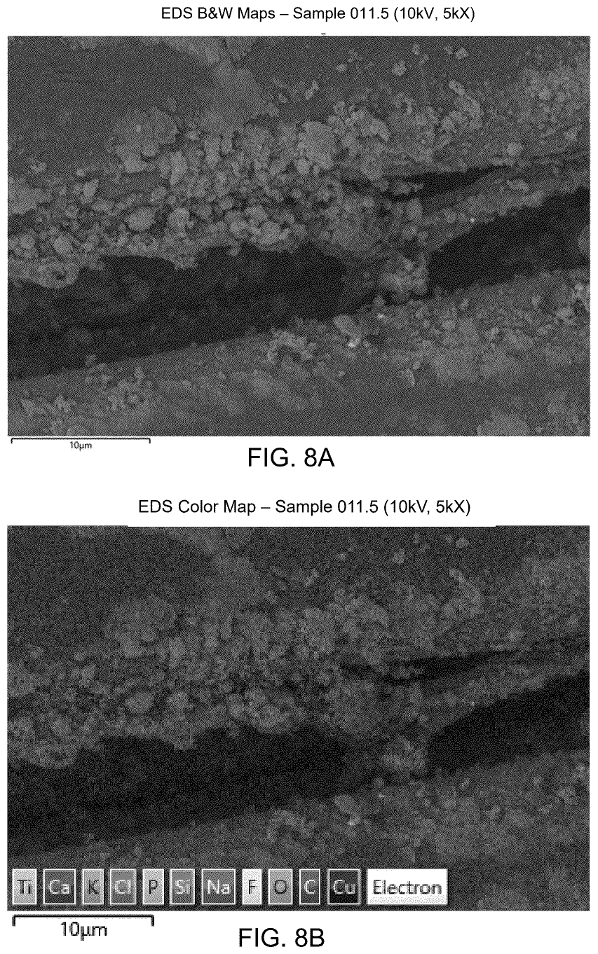

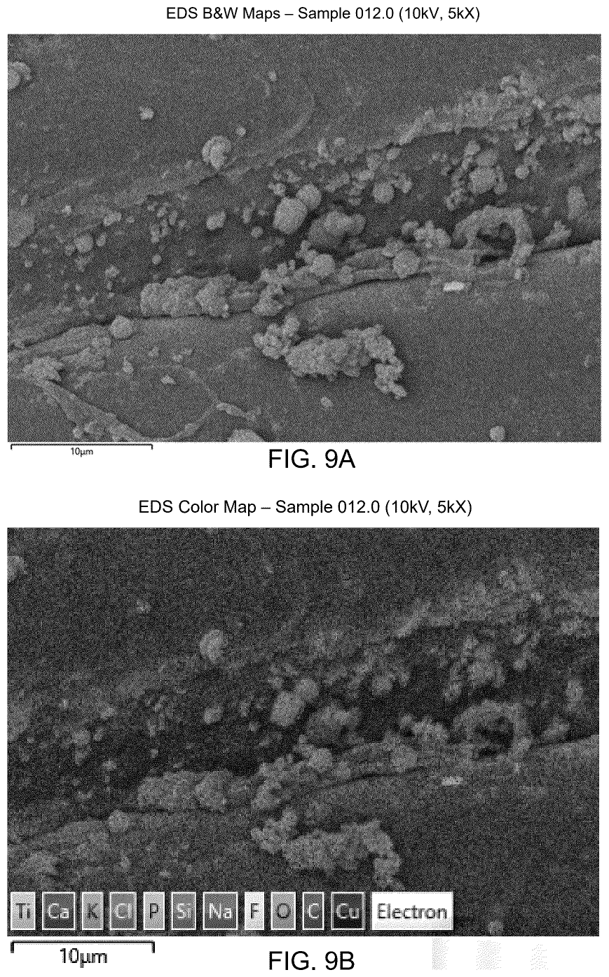

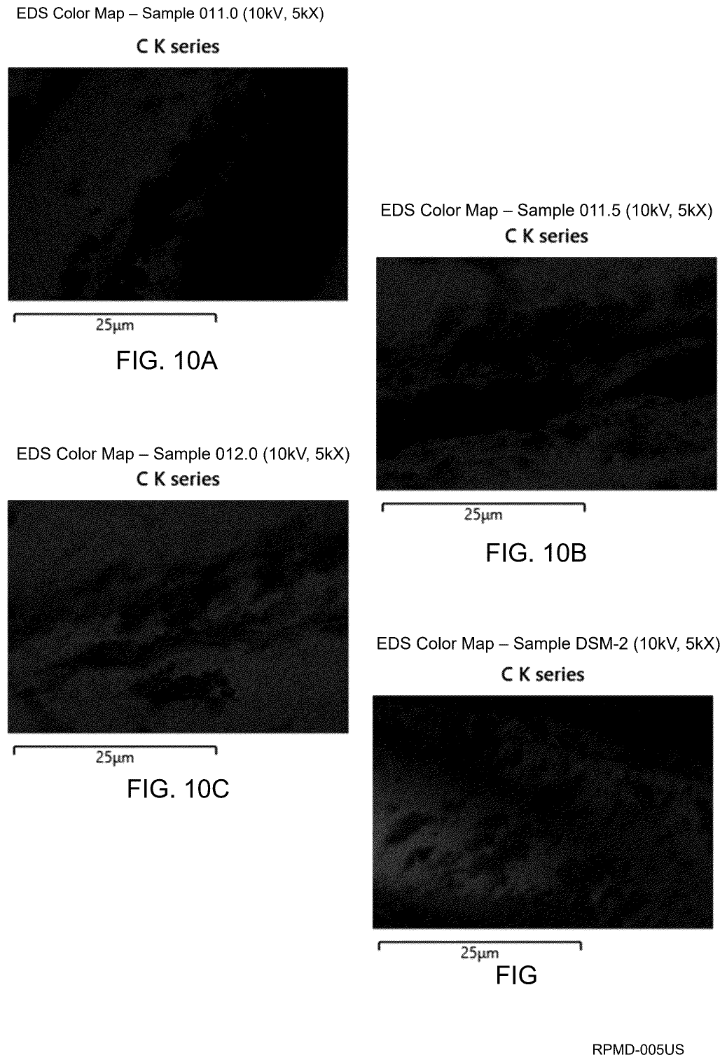

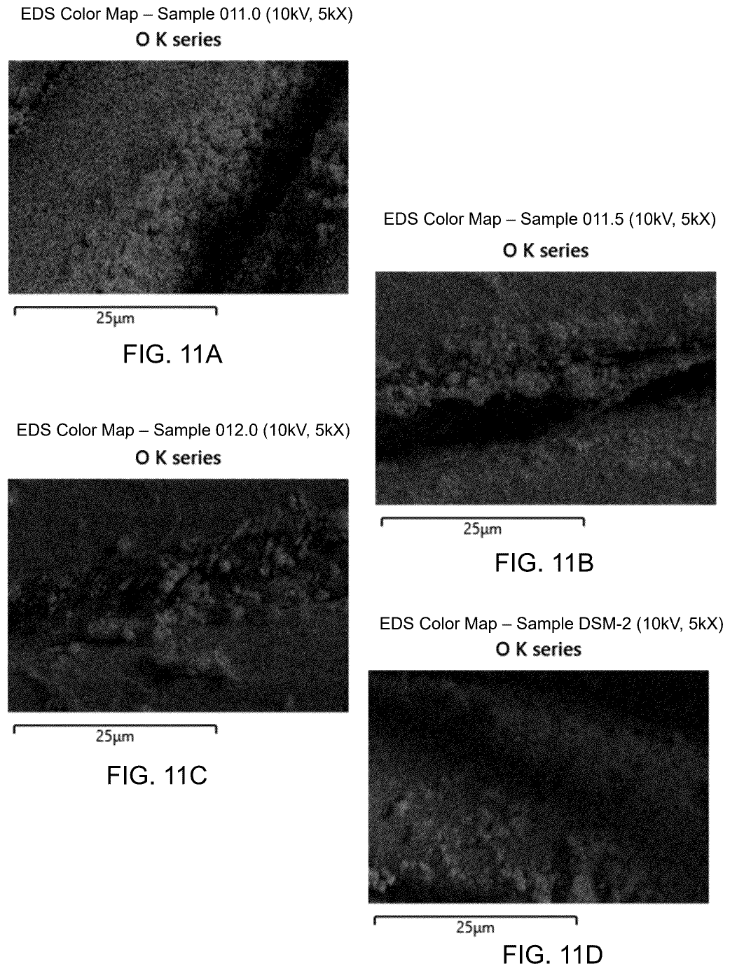

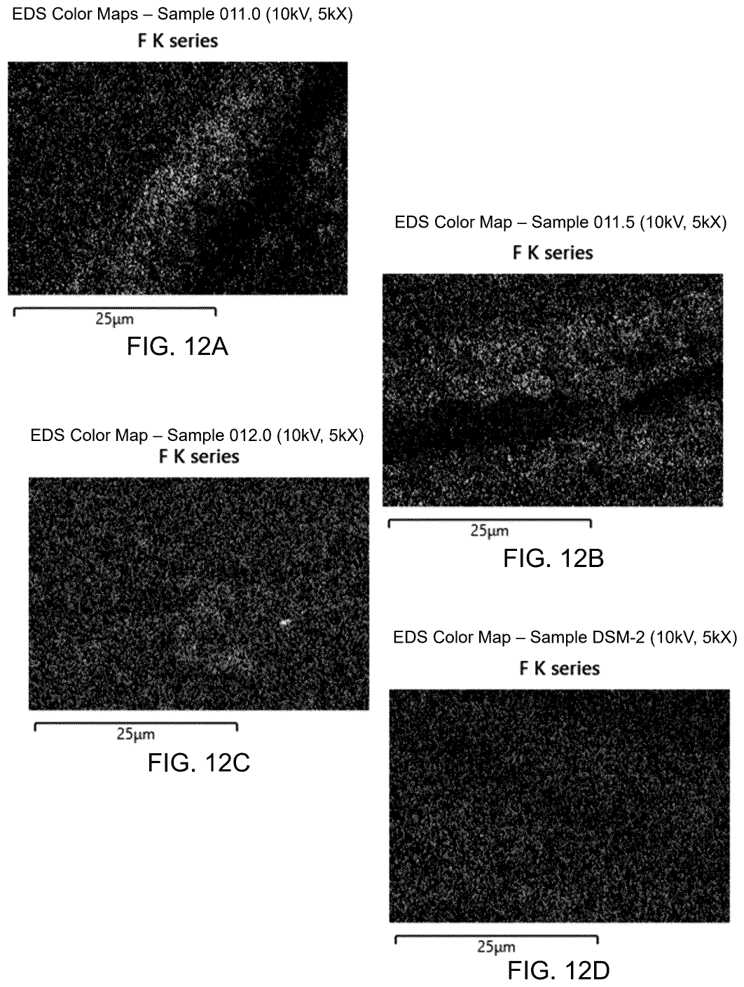

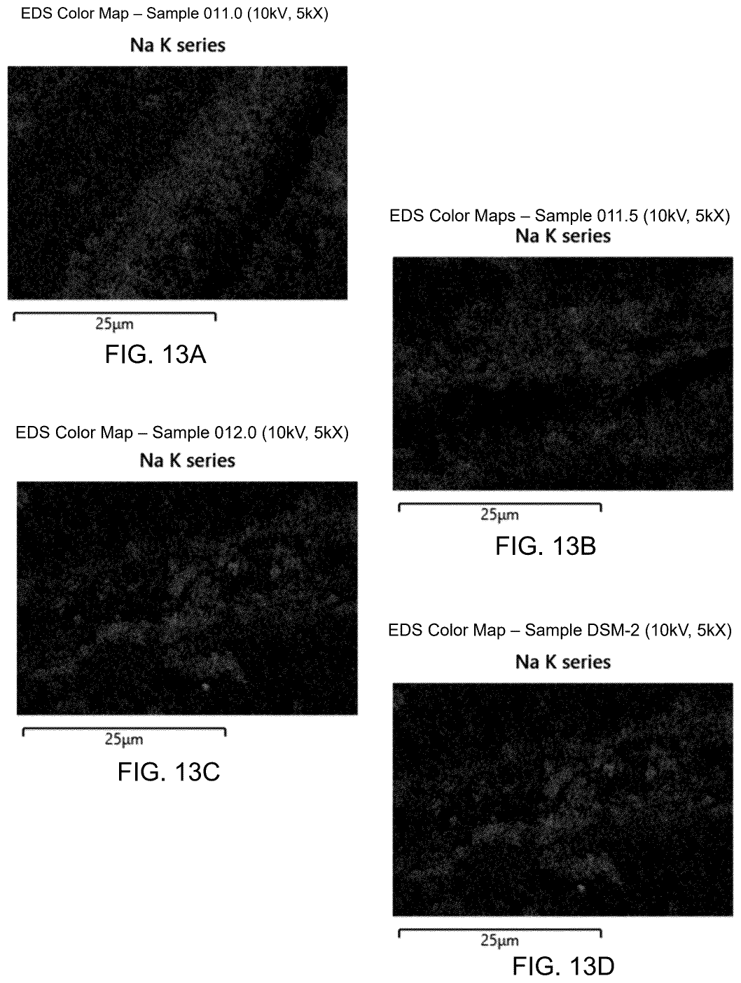

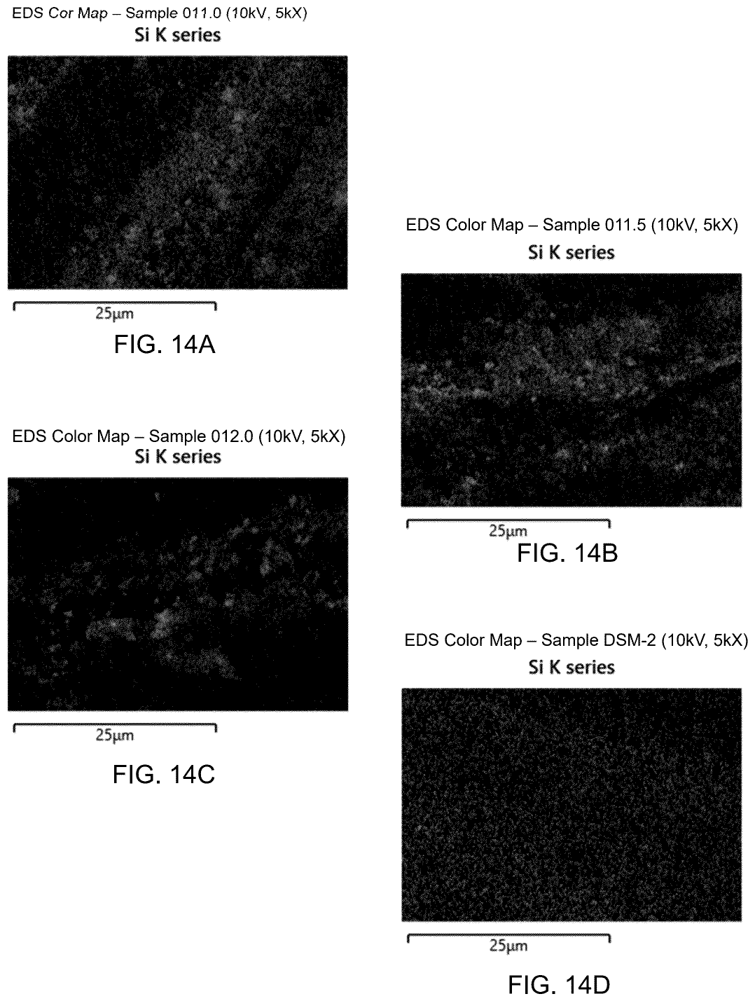

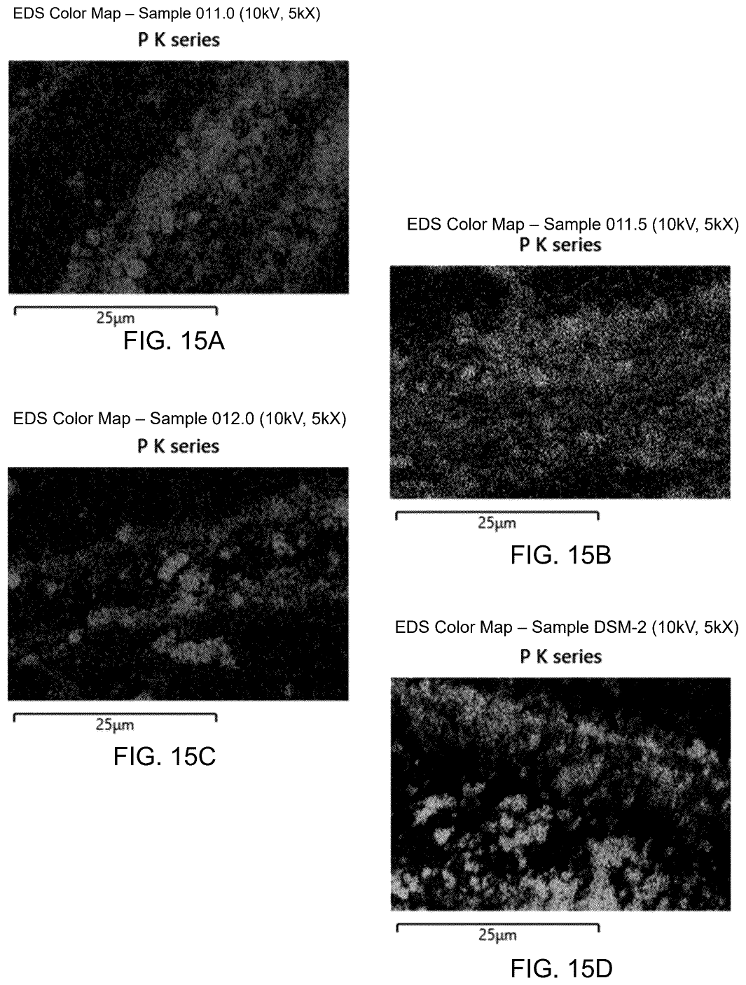

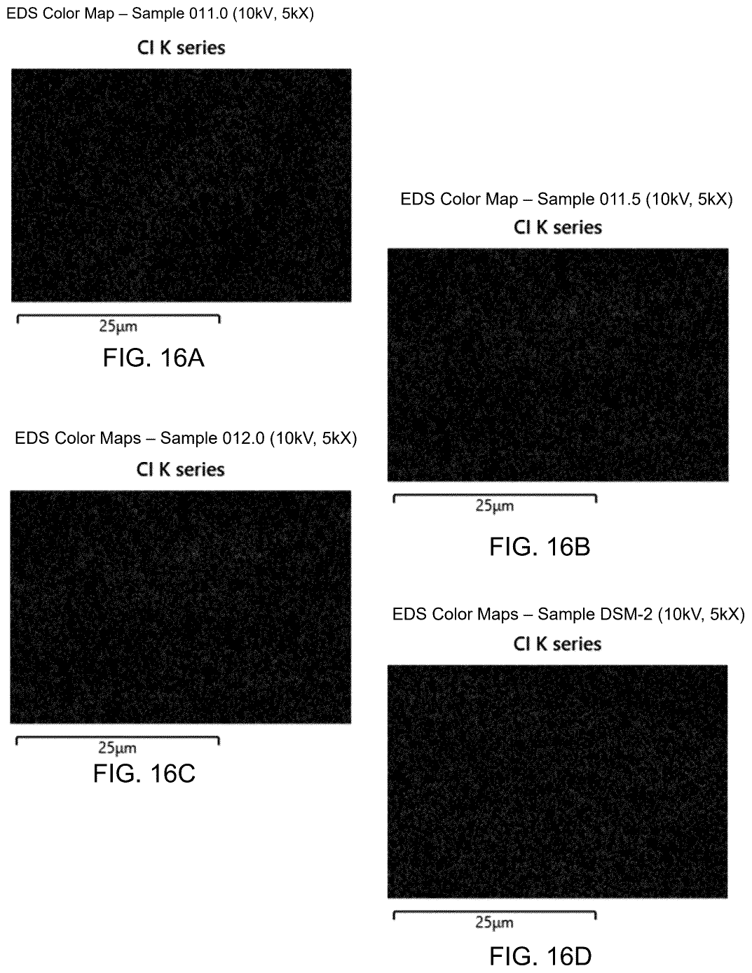

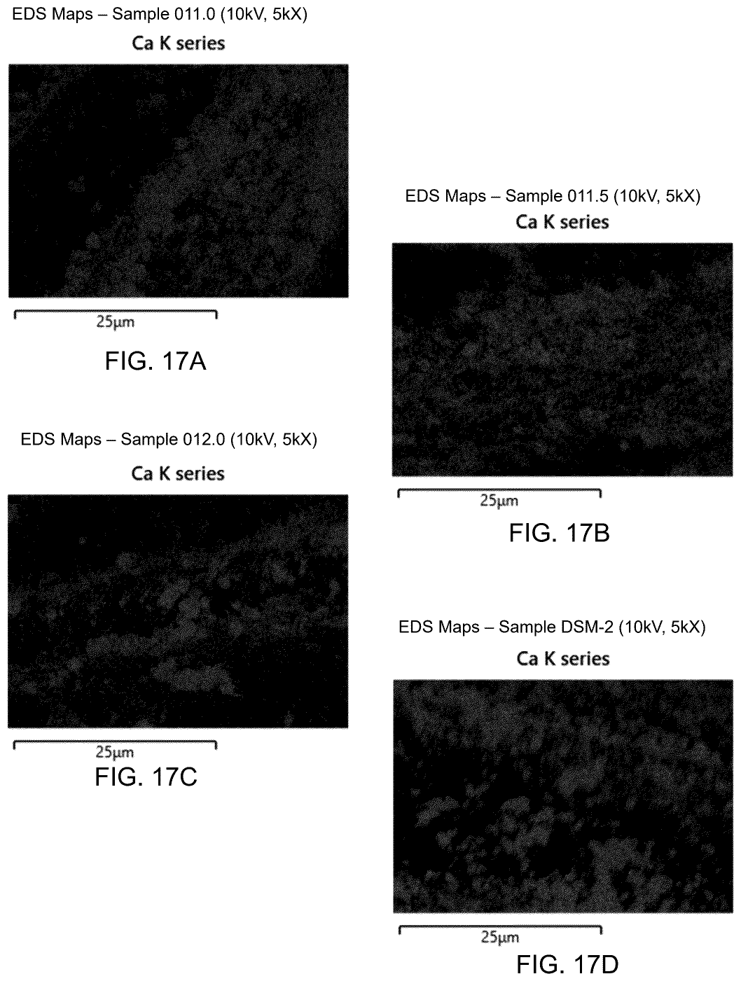

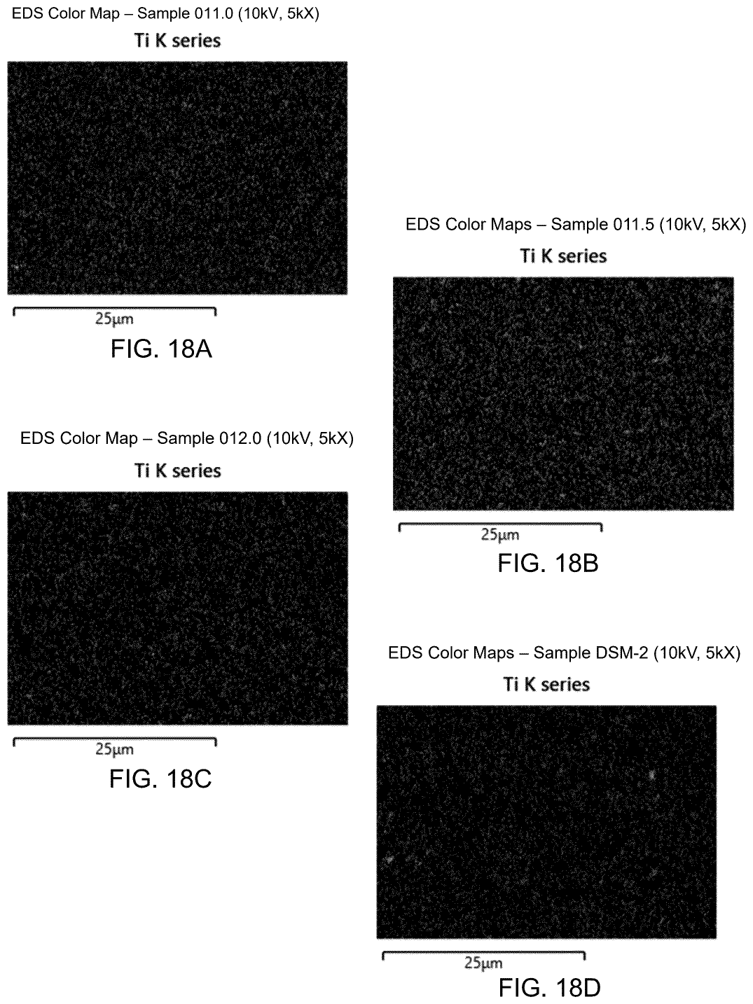

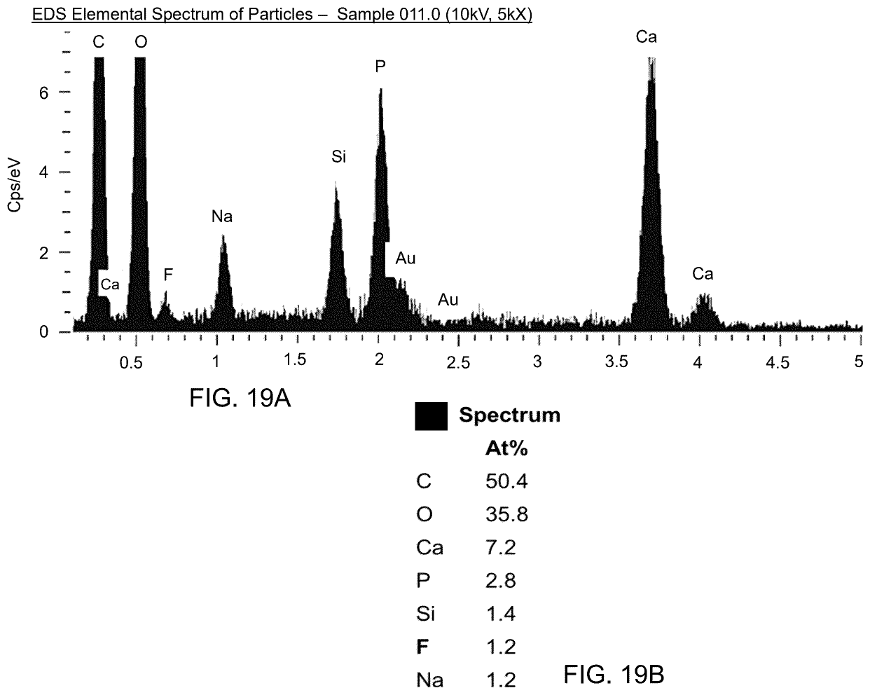

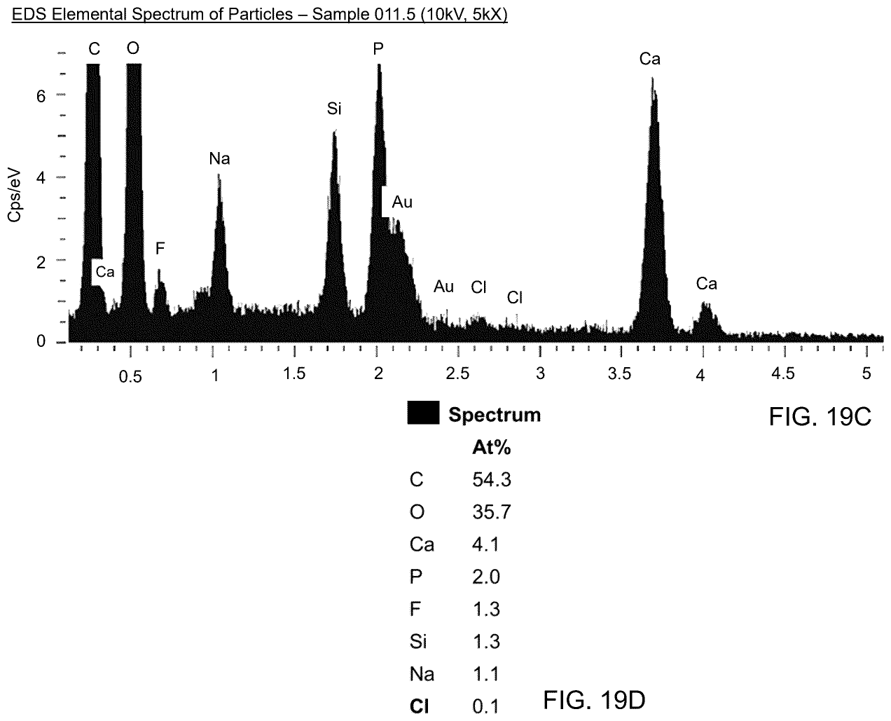

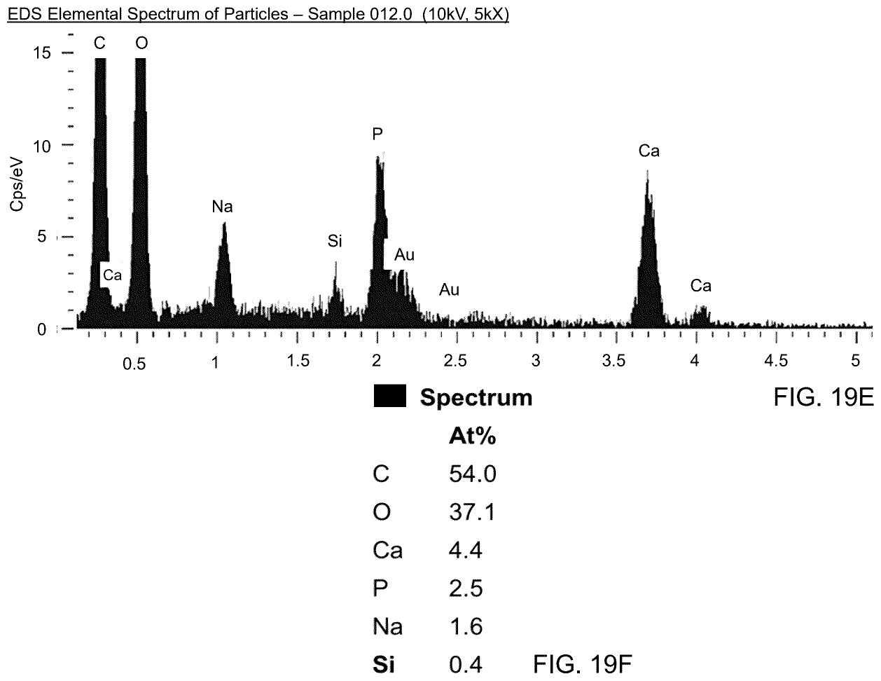

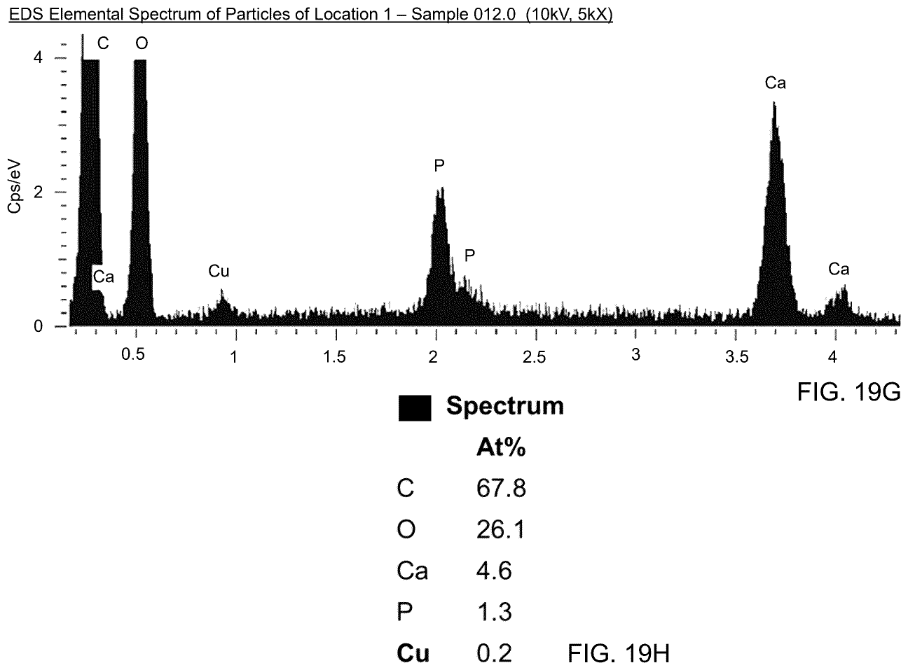

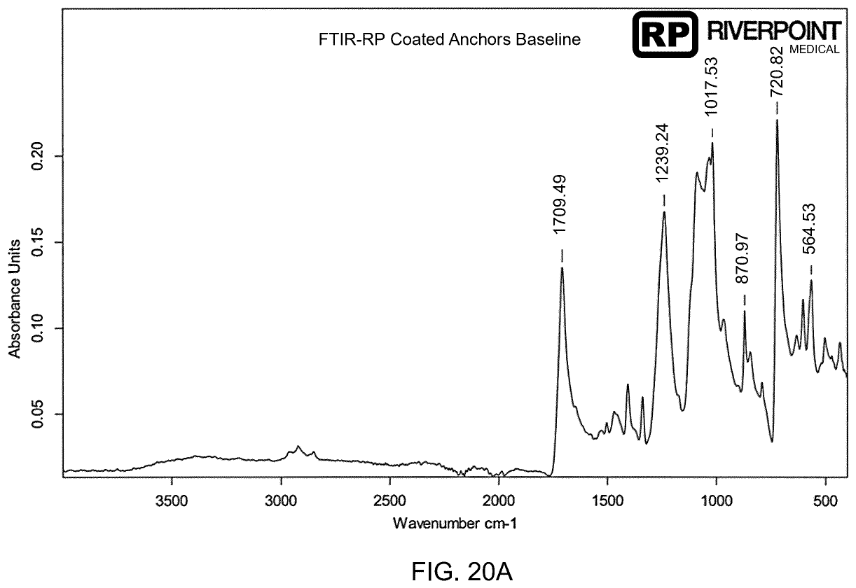

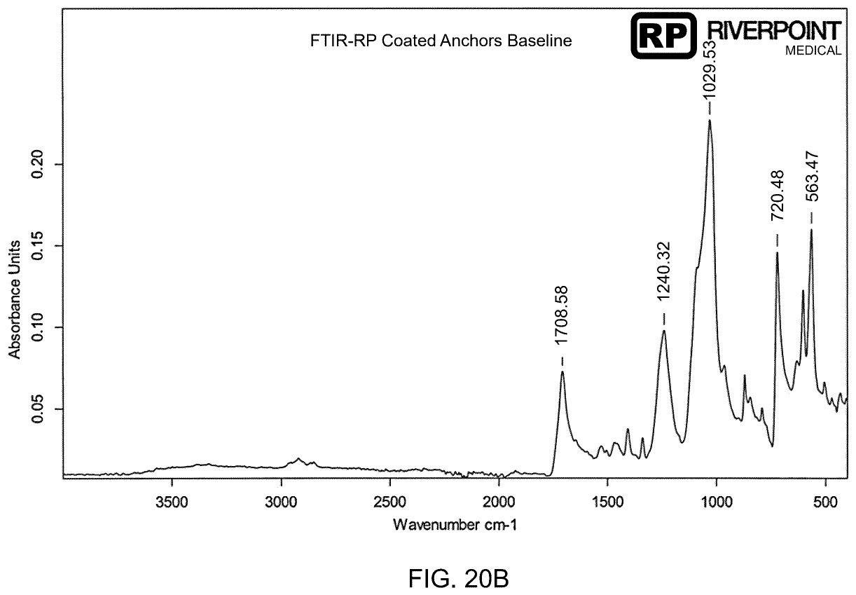

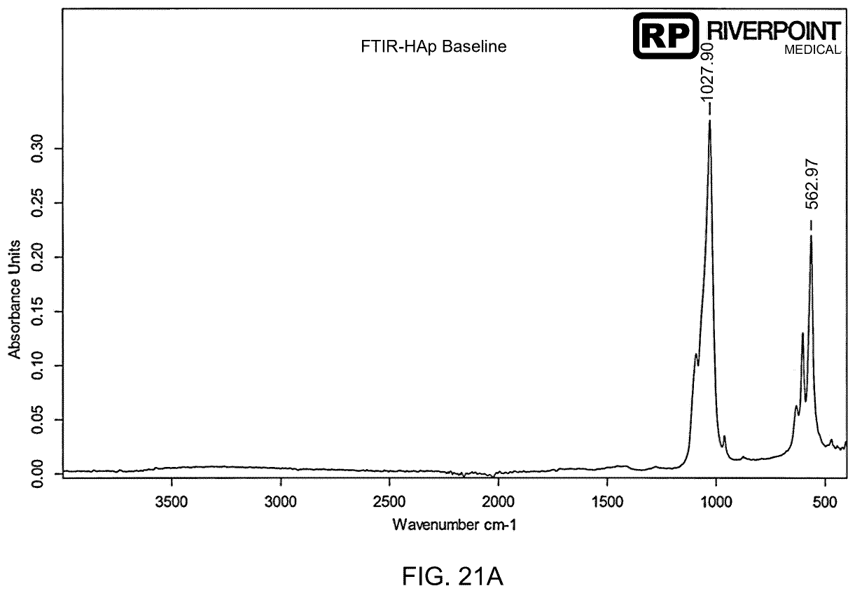

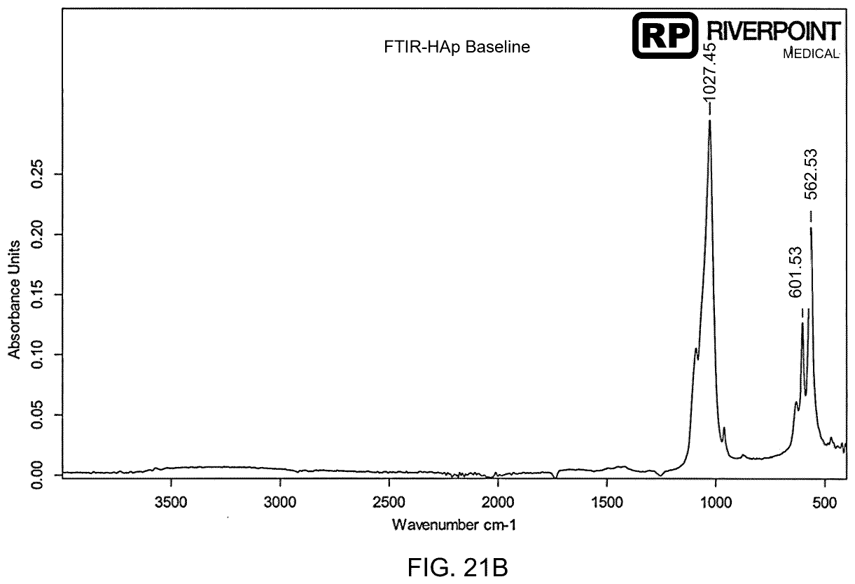

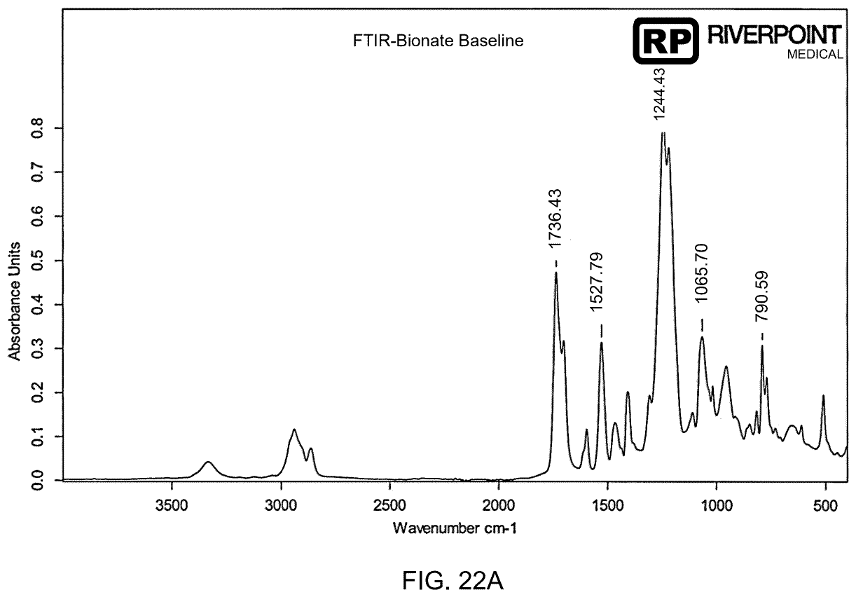

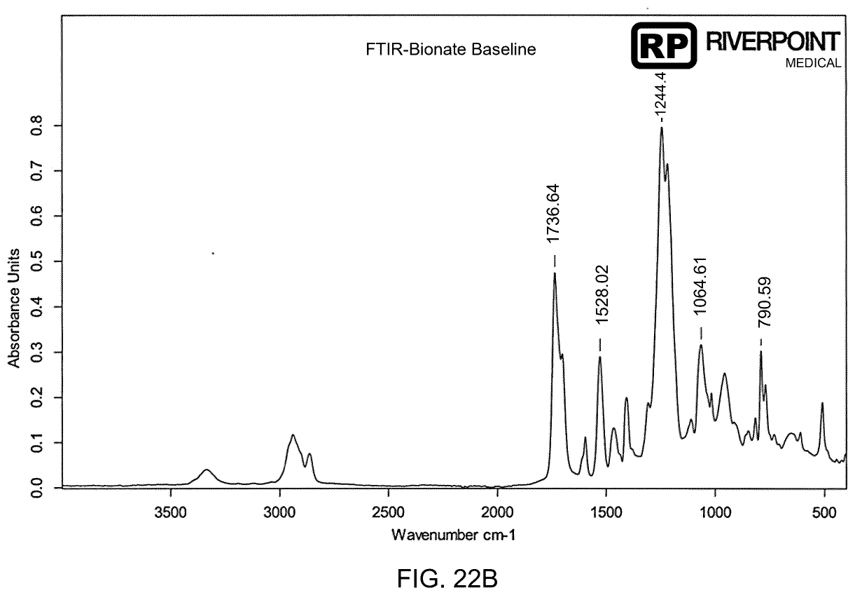

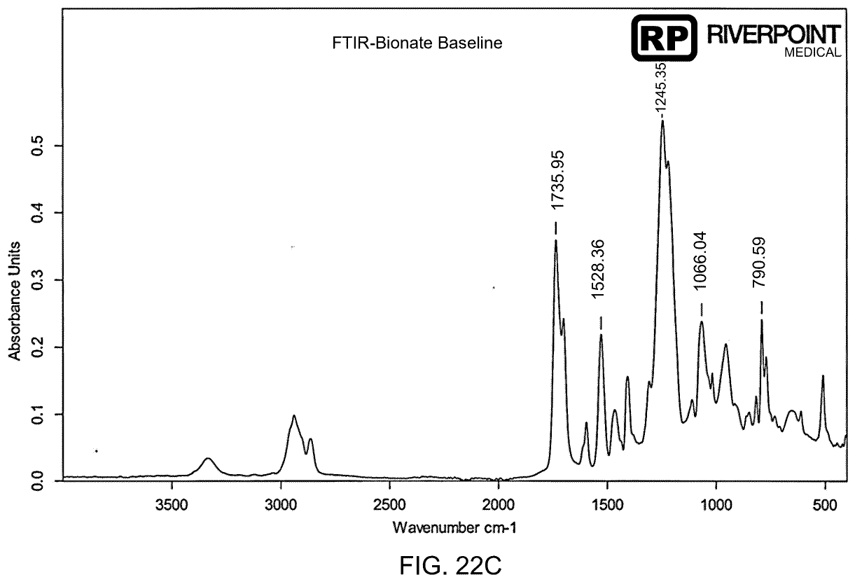

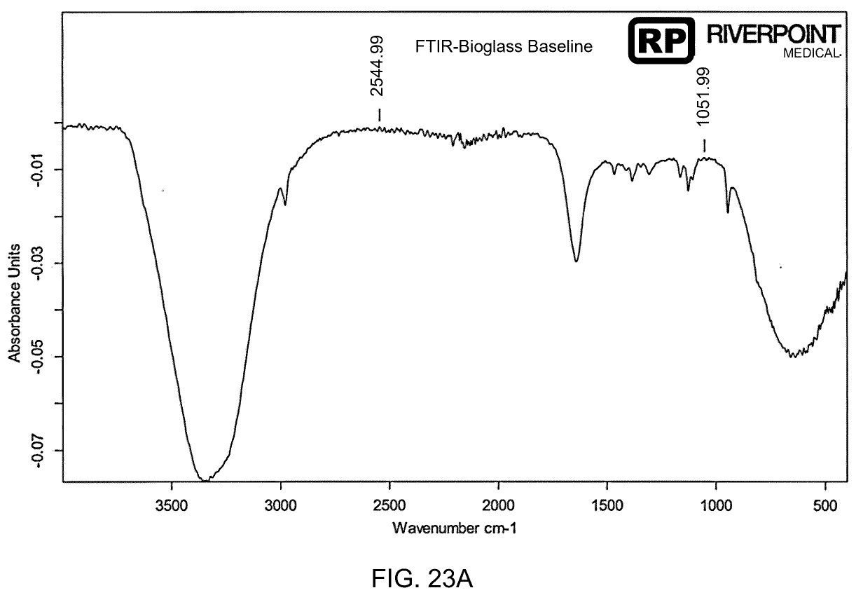

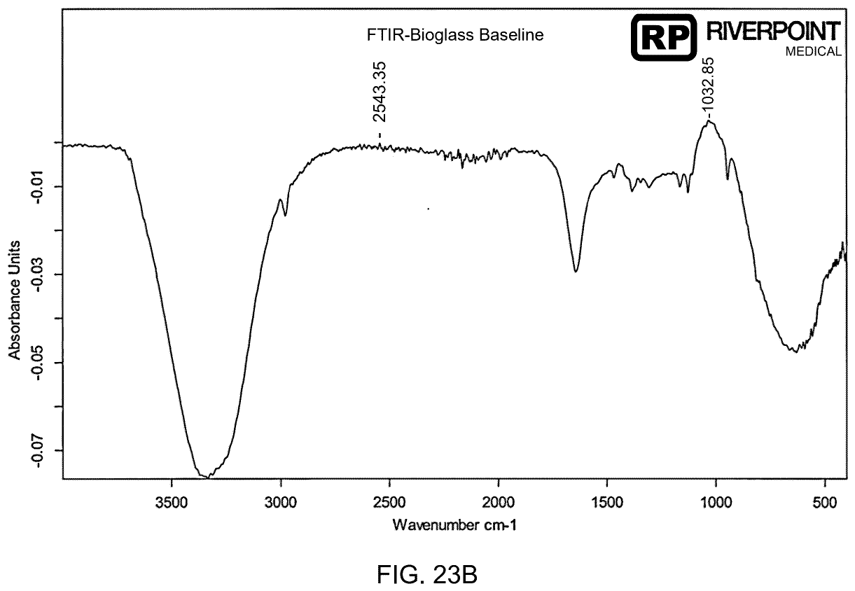

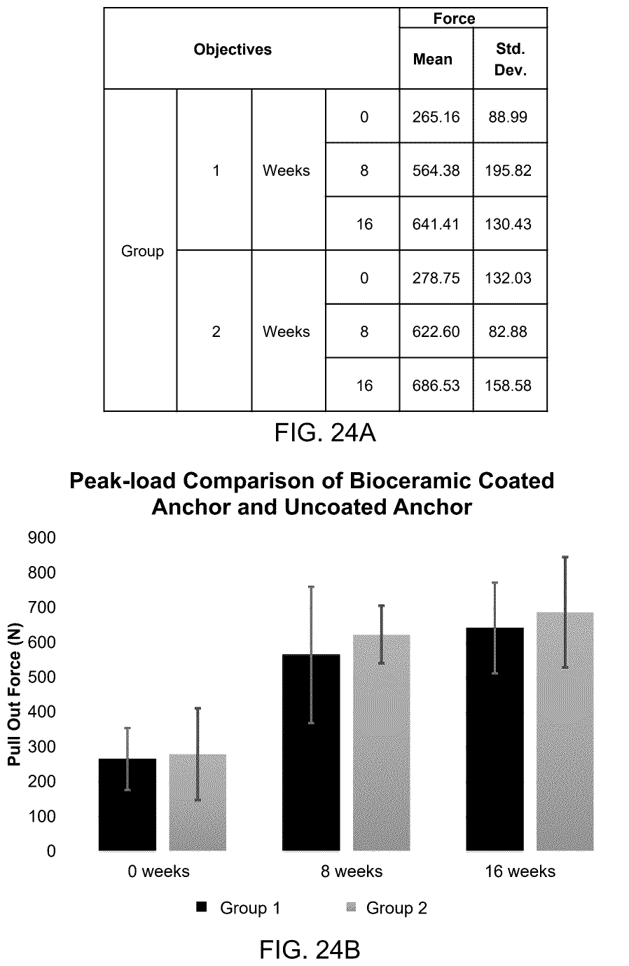

With reference to FIGS. 7A-7B, 8A-8B, 9A-9B, the images illustrate EDS Black & White (B&W) and the EDS color overlay maps for Samples 011, 011.5 and 012. EDS enables the chemical characterization and/or molecular analysis of materials. EDS functionality may be integrated with an SEM instrument to provide EDS/SEM layered images for qualitative analysis of the compositional elements of one or more bioceramic particles at specified or predetermined site locations on the coated product. The EDS spectrum of molecules plots display data ax s-ray counts vs. energy (in keV), and the energy corresponds to the various elements in the sample in at % or wt. %. Furthermore, EDS phase color maps may help determine areas with aggregate molecules with different compositions, providing each composition or assigning each composition a different color. This allows a person to visualize the density of each of the compositional elements of the bioceramic particles disposed onto a coated product, as well as any contaminants. For example, FIG. 7B illustrates each compositional element located on the surface of a bioceramic coated product and assigns a unique identifiable color that will be highlighted on the map. The identified compositional elements include Ti, K, F, Ca, CL, P, Si, Na, O, and C. EDS maps or EDS analysis may also be converted to 3D surface plots to help determine the surface roughness or height of each coating (not shown). FIGS. 10A-10B, 11A-11D, 12A-12D, 13A-13D, 14A-14D, 15A-15D, 16A-16D, 17A-17D, 18A-18D are EDS colored maps for Samples 011, 011.5 and 012 display the density of each isolated compositional element of the bioceramic coating into its identifiable color.

With reference to 19A-19H, 20A-20B, 21A-21B, 22A-22C and 23A-23B, illustrate EDS elemental spectrum maps and FTIR maps that identify the compositional elements of the bioceramic particles and assigns a quantitative atomic weight % (at %) or absorbance units. A specific surface area and magnification is selected, and used to obtain the quantitative elemental values. The FTIR applies infrared radiation to the one or more coated products and measures the absorbance of infrared light at various wavelengths to determine the composition and or molecules of the product. The x-axis represents the infrared spectrum, which pots the intensity of the infrared spectra, and the y-axis represents the amount of infrared light absorbed or transmitted by the material(s) being analyzed. One or more of the infrared wavelengths absorption peaks identifies the desired molecules of Si, Na, Ca, P and/or N.

The mechanical properties may be performed by a variety of different analytical techniques, including adherence of each coating, coated product flexibility, coated product tensile strength, coated product elongation, coated product knot breakage, accelerated aging, and/or any combination thereof. Such analytical techniques for testing of mechanical properties may be performed by standard ISO procedures, internal company testing procedures, and/or desired customer procedures. Furthermore, prior to the analysis of each one or more samples of the coated products may require initial preparation.

The biological implant properties may include bioactive and/or healing responses properties. Such bioactive responses may include osteointegration or osteostimulation response, antimicrobial response, antibacterial response, wound healing response, angiogenic response, tissue regeneration response, hemostatic response, a delivery or carrier response and/or any combination thereof. In one embodiment, the bioactive response may include an osteogenic or osteostimulation response. Such osteogenic or osteostimulation healing response may include properties of implantation pull-out strength, implant geometry of the coated product, implantation bone reactions, implantation tissue reactions, implantation osteointegration, implantation inflammatory biomarkers, implantation pH, implantation bone tunnel diameter, implantation migration (change in position) and/or any combination thereof. Collection of such biological implant properties or healing response properties may be performed by various analytical techniques, including Instrons or MTS MiniBionix, MicroCT, radiograpy, histology, histophometry, bloodwork, periotest, percussion test, reverse torque test, resonance frequency analysis (RFA) to determine the Osstell ISQ scale and/or any combination thereof. The successful bioactive or healing response may be equal to or superior to uncoated products.

In one exemplary embodiment, the bioactive response may include an antimicrobial response. The implantation of bioactive glass (1) exposes the bioactive glass granules to physiological or body fluids to cause the release or exchange of network-modifier ions (e.g., Si, Na⁺, K⁺, Ca²⁺ with H⁺ or H₃O⁺ ions) from surrounding body fluids. This reaction gives rise to an increase in osmotic pressure forcing microbial disadvantageous morphological changes and an increase in the local pH from 7 to 11, resulting in an alkaline microenvironment. Additionally, even the physical characteristics of the bioactive glass—i.e., the “needle-like” sharp glass—that could potentially damage microbe cell walls, thus creating hollows or holes to facilitate the penetration of antimicrobial agents in the microbial cytoplasm. These mechanisms of action (increase of pH, increase osmotic pressure and physical destruction) efficiently inhibit microbial growth and/or microbial biofilms. Such antimicrobial response may include properties such as time observed for an initial increase in pH (10 hours or less); total time to maintain the increase of pH (greater than or equal to 4 hours or 4 hours to 36 hours), increase in local pH (a range of 7 to 11); concentration of one or more solutes or released ions in the body fluid (e.g., Si, Na⁺, K⁺, Ca²⁺ with H⁺ or H₃O⁺ ions); concentration of one or more solutes or released ions in the microbial cytoplasm (e.g., Si, Na⁺, K⁺, Ca²⁺ with H⁺ or H₃O⁺ ions); maximum or minimum particle size for antimicrobial effect (at least 10 μm or greater or a range of 20 μm to 140 μm); reduction in inflammatory biomarkers; a reduction in infection biomarkers; percent of microbial cell viability; and/or in vitro cytotoxicity. The successful antimicrobial bioactive or healing response may be equal to or superior to uncoated products.

For example, in one exemplary embodiment, bioactive response comprises an osteogenic response. The osteogenic response includes osteointegration. Osseointegration is defined as a direct bone anchorage or integration to an implant which can provide a foundation to support a prosthesis and provide stability. Implant stability is a requisite characteristic of osseointegration. Without it, long-term success cannot be achieved. Continuous monitoring in a quantitative and objective manner is important to determine the status of implant stability. Osseointegration is also a measure of implant stability which can occur in two stages: Primary and secondary. Primary stability mostly occurs from mechanical attachment with cortical bone. Secondary stability offers biological stability through bone regeneration and remodeling. Primary stability is affected by bone quality and quantity (e.g., implantation bone reactions), surgical technique, pull-out strength, and implant geometry (length, diameter, surface characteristics) (e.g., implantation geometry). Secondary stability is affected by primary stability. The successful osteogenic bioactive or healing response may be equal to or superior to uncoated products.

The histology or histomorphometric analysis obtained by calculating the peri-implant bone quantity and bone-implant contact (BIC) from a dyed specimen of the implant and peri-implant bone. It is assessed at pre-, intra-, and post-surgical time points. The push-out/pull-out test investigates the healing capabilities at the bone implant interface. It measures interfacial shear strength by applying load parallel to the implant-bone interface. In the typical push-out or pull-out test, a cylinder-type implant is placed transcortically or intramedullarly in bone structures and then removed by applying a force parallel to the interface. The maximum load capability (or failure load) is defined as the maximum force on the force displacement plot, and the interfacial stiffness is visualized as the slope of a tangent approximately at the linear region of the force displacement curve before breakpoint. It is assessed during the healing period of the one or more coated products.

Furthermore, imaging techniques such as Micro CT and radiography, are widely used to assess both quantity and quality of the bone. The imaging techniques are used to assess the health of the implant, evaluating the bone quantity and quality changes, and estimating the crestal bone loss, changes in bone mineral, which is a consequence of the osseointegration process.

Accordingly, the raw data collected from the characterization methods may be used to establish one or more QC inspection criteria. The QC inspection criteria can then be used to approve one or more bioceramic coated products that can predict or correlate with a specific healing response, e.g., a bioactivity or biocompatible response.

As disclosed herein, the method of predefining a set of QC acceptance criteria comprises the step of: generating one or more QC acceptance criteria data using the raw data 24. With reference to FIGS. 2 and 3D, the tables disclose a plurality of characterization tests or techniques which raw data may be collected and statistically analyzed to provide one or more quality control acceptance criteria that provides a sufficient confidence that the one or more bioceramic coated products will provide or generate an enhanced or optimized healing response once implanted (e.g., enhanced bioactivity or biocompatible response) that will desirably improve clinical outcome.

In one exemplary embodiment, the one or more bioceramic coated products comprises a QC acceptance criterion for bioceramic coating consistency and/or homogeneity. The QC acceptance criteria comprises a homogeneity osteoconductive (OC) ratio. The homogeneity OC ratio comprises the sum of the compositional elements of the bioceramic coating of the one or more bioceramic coated products from at least one site location, N (number of site locations), using SEM-EDS imaging technique. The homogeneity ratio for the at least one site location includes the sum of Ca+Na+P+Si as collected in weight % (wt. %). Accordingly, it is desired that the homogeneity OC ratio comprises the sum of the compositional elements of the bioceramic coating of the one or more bioceramic coated products from a plurality of site locations using SEM-EDS imaging technique. The average homogeneity ratio for the plurality of site locations includes the sum of [Ca+Na+P+Si as collected in weight % (wt. %) at each of the plurality of site locations]/(N) (the number of site locations).

As disclosed herein, the method for quality control inspection of bioceramic coated products with enhanced or controlled bioactive and/or biocompatible properties (e.g., healing response) comprises the step of: verifying, confirming or approving the sample data meets the QC acceptance criteria 16. The sample data will be representative of at least one lot of one or more coated products manufactured to set or standardized manufacturing procedures. The sample data collected from the characterization step will also provide the proper or desired summary data resulting from the one or more characterization methods. The sample data will be compared to the QC acceptance criteria to present a “pass” or “fail” of the at least one manufactured lot to ensure reproducibility and consistency between the manufactured lots. This step 16 may comprise the approving step 18 disclosed herein below.

As disclosed herein, the method for quality control inspection of coated products with enhanced or controlled bioactive and/or biocompatible properties (e.g., healing response) comprises the step of: approving the at least one manufactured lots of the one or more coated products having an optimized or enhanced healing response 18. If the manufactured lot of the one or more coated products “passes,” then the manufactured lot of the one or more coated products may proceed to packaging and/or sterilization. If the manufactured lot of the one or more coated products “fails,” then the manufactured lot of the one or more coated products may be destroyed, and further investigation on the failed QC criteria and its relative manufacturing step should be required. The approving step 18 may be part of merged with the confirming step 16.

### EXAMPLES AND EXPERIMENT DATA

### Bioceramic Coated Products

In one embodiment, the one or more bioceramic coated product comprises: a textile product including a textile material, the textile material comprises a biocompatible, non-biodegradable polymer material; a first coating layer including a first coating material and a first coating solvent, the first coating material comprises a biocompatible, non-biodegrable polymer material; and a second coating layer, the second coating layer comprises one or more bioceramic particles and a second coating solvent. The one or more bioceramic particles comprises Bioglass®. The one or more bioceramic particles comprises Bioglass® and HA.

In another embodiment, the method of making one or more bioceramic coated products comprises the steps of: obtaining, providing or manufacturing a textile product including a textile material, the textile material comprises a biocompatible, non-biodegradable polymer material; depositing a first coating layer onto a outer surface of the textile product, the first coating layer including a first coating material and a first coating solvent, the first coating material comprises a biocompatible, non-biodegrable polymer material; and depositing a second coating layer onto a portion of the first coating layer, the second coating layer comprises one or more bioceramic particles and a second coating solvent. The one or more bioceramic particles comprises Bioglass®. The one or more bioceramic particles comprises Bioglass® and HA.

### Coated Product Preparation

The textile product comprises an all-suture anchor. The textile product included braided construction made from a textile material of multi-filament PET yams. At least two all-suture anchors were provided having a diameter of 1.4 mm and a 2.3 mm, and a length of at least 30 to 50 mm. The denier for the all-suture anchors ranged from 300 denier to 600 denier. The PPI for the all-suture anchors ranged from 15 PPI to 40 PPI. All textile products were cleaned, pre-wetted or pretreated before further testing in THF for up to 3 min.

Coating Textile Products with First and Second Coating Layer

The first coating layer comprises a dispersion or solution of Bionate® PCU 80A, a thermoplastic polyurethane was prepared and diluted with THF. The textile products were coated with Bionate by dipping the textile product in the first coating layer solution for a period of time. The first coated layer textile products were dried to evaporate the THF. After drying the first coated layer textile products were dried, they were dipped into the second coating layer dispersion or solution. The second coating layer dispersion comprises hydroxyapatite (HA) and Bioglass in a second coating solvent, THF. The first and second coated layer textile products were dried before testing.

### In-Vivo & In-Vitro Testing

This study is designed to evaluate the roles of surface treatment and implant topography on early bone ongrowth/ingrowth and functional osseointegration. The specific objectives of the study were: To evaluate the radiographic appearance cancellous implantation sites, mechanical properties in cancellous implantation sites, quantify the amount of osseointegration cancellous implantation sites at 0, 8, and 16 weeks.

The one or more bioceramic coated textile products (Group 2) and uncoated textile products (control, Group 1) were implanted into the cancellous bone bilaterally in the distal femur and proximal tibia of an animal model. There were 3 implantations in the femur and 1 on the tibia on the right and left sides (8 per animal). The remaining sutures on the one or more bioceramic coated textile products and uncoated textile products were shuttled into a subcutaneous pocket for harvesting to allow mechanical pullout testing. The tissues were reflected, and the skin closed with resorbable sutures. Once the procedure was complete, the animals were recovered according to standard procedures. All of the one or more bioceramic coated textile products and uncoated textile products (control) were properly evaluated at 0, 8 and 16 weeks as the objectives recited.

### Osteointegration Via Pull-Out Force

Cyclic and Pull-out testing were performed at a figure gauge length using a calibrated MTS Mini Bionix®. All of the one or more bioceramic coated textile products and uncoated textile products (control) was fixed with suture grips and the bone rigidly fixed. Testing was performed at 100% per min (gauge length 30 mm). Peak Load, Stiffness, and Energy were reported and used for statistical analysis using a 2-way ANOVA (Group and 1).

The one or more bioceramic coated textile products (Group 2) showed an increase in pull-out forces at 8 and 16 weeks over the uncoated textile products (Group 1) after the initiation of the osteointegration process. At 8 weeks and 16 weeks, Group 2 shows at least a 7% to 11% change or greater change over Group 1.

### Osteointegration Via Bone Formation and Surrounding Tissue Reactions

PMMA histology and histomorphometry was used to evaluate the cancellous specimens surrounding Groups 1 and 2. Collected cancellous specimens were prepared to be embedded in polymethylmethacrylate (PMMA). Embedded and cancellous implants were sectioned or sliced along the long axis of the implants using a Leica SP 1600 Microtome. The slices were stained, resulting in bone staining pink, fibrous tissue blue/purple. Slides were examined under a light microscope (Olympus, Japan) for bone integration and reaction at the implant-bone interface, including general tissue response, the presence of inflammatory cells or local particulate.

The PMMA images were taken at the bone implant interfaces for determination of bone ingrowth/bone ongrowth following histomorphpmetry procedures. Low magnification images (1.25×, 1 mm scale bar) were taken from to assess the interface of the bone and implant interface. The regions of interest (ROI) for each image identified and evaluated.

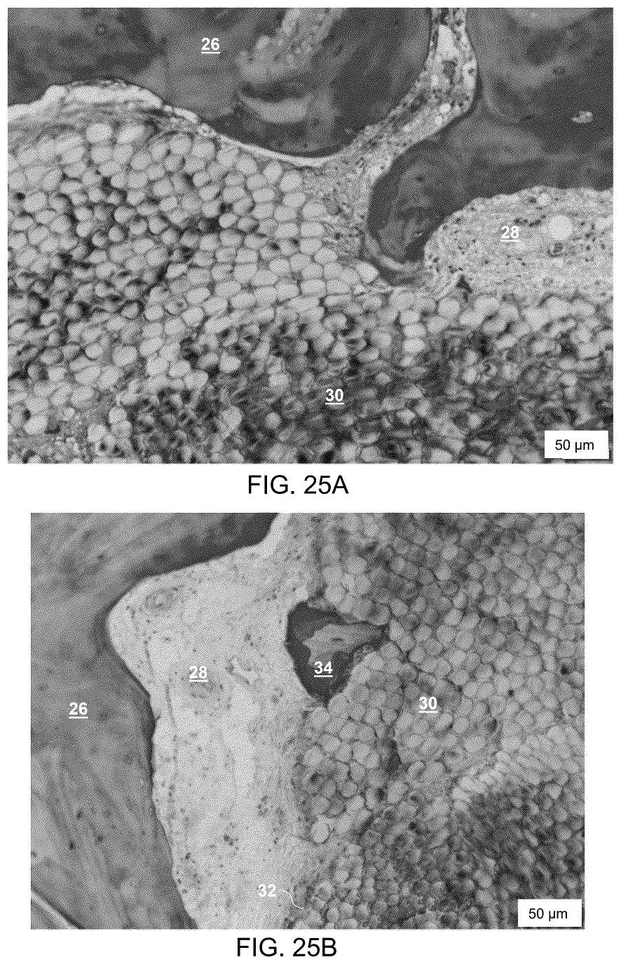

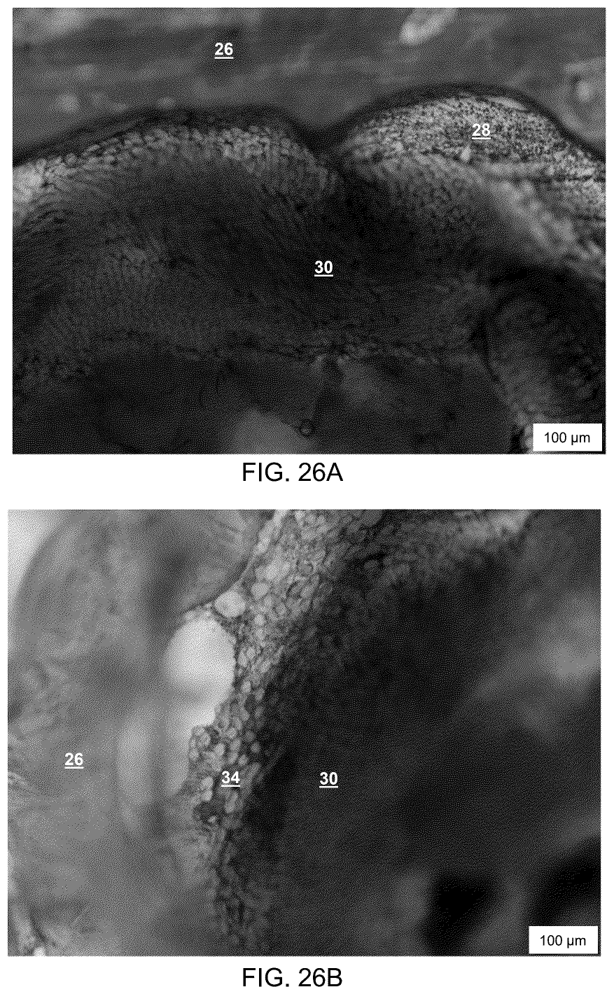

PMMA histology histomophometry at 8 and 16 weeks for groups 1 and 2 did not reveal any adverse environmental reactions within the cancellous bone 26 implantation sites consistent with the micro-computed tomography data as shown FIG. 25A (Group 1, 8 weeks), FIG. 25B (Group 2, 8 weeks), FIG. 26A (Group 1, 16 weeks) and FIG. 26B (Group 2, 16, weeks). The multinucleated cells (macrophages) were found on the surface of the coated and uncoated textiles sutures 30 in both groups. The immediate adjacent marrow spaces were normal. The interface of the textile sutures 30 between Groups 1 and 2 and the host bone demonstrated new bone formation 32 as the bone healed in direct contact with both devices, as well as some fibrous tissue 28. New bone formation 32 on the interface of the Groups 1 and 2 was observed at 8 weeks has remodeled at 16 weeks. However, Group 2 consistently showed new bone formation as compared to Group 1, and Group 2 showed new integrated bone formation 34 at 8 and 16-weeks directly on the material and integrated with into the textile sutures 30 as shown in FIG. 26B.

### Establishing the Bioceramic Coating Homogeneity & Repeatability Ratio

SEM images, SEM-EDS color maps and SEM-EDS spectrum maps were obtained to determine coating variability and/or homogeneity of the bioceramic coating on the one or more coated products, and respectively a minimum QC acceptance criterion for the homogeneity osteoconductive (OC) ratio. The average homogeneity OC ratio was calculated using (Na+Ca+P+Si)/N (number of site locations).

Using the active ingredients or molecules in the bioceramic coating facilitates the accurate identification of the active molecules on the one or more coated products. As disclosed herein, the active ingredients are molecules are significant and directly relevant to the ionic exchange (e.g., Si, Na⁺, K⁺, Ca²⁺ with H⁺ or H₃O⁺ ions) that occurs once the bioceramic coating is exposed to surrounding body fluids to initiate and form an HA layer. Furthermore, the ionic dissolution leads to upregulation of several gene families involved in osteogenesis, antimicrobial and angiogenesis activities. Therefore, the identification of a minimum amount of the active ingredients or molecules gives rise or relates to the homogeneous coverage, uniform coverage or surface area coverage intended to produce an effective bioactive or healing response (e.g., there should be enough active ingredients or molecules on the surface of the coated products to initiate the ionic exchange to form the HA layer and subsequent cellular or gene responses for success osteogenic, antimicrobial and/or angiogenic activities).

At least four individual bioceramic coated textile products were sent to an external laboratory. At least four (4) site locations on each of the bioceramic coated textile products were evaluated, resulting in 16 data points that were statistically analyzed. The calculated average was 5.61 wt %. The calculated standard deviation was 0.9175. The 90% CI of the standard deviation was [0.711, 1.319]. The lower CI and z-value of 1.282 was used to calculate the wt % of 4.7. Based on the results, the minimum homogeneity OC Ratio for the 90% Cl is set to 4.7%.

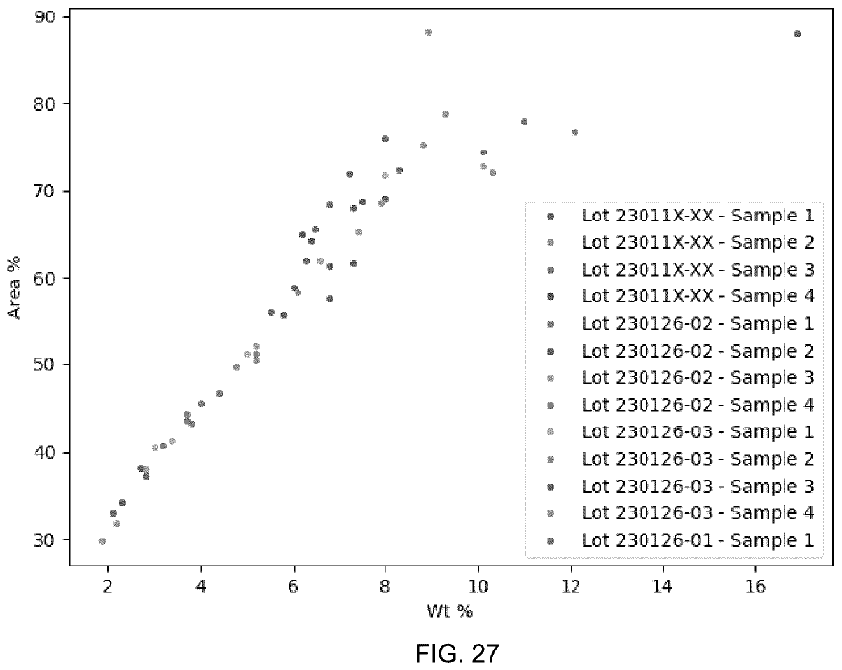

Furthermore, the raw OC ratio data collected was used to determine the strength of relationship between the OC ratio and the total percent coverage area of the bioceramic coating. FIG. 27 shows or illustrates an unexpected, but strong or positive linear relationship between the OC ration and total percent coverage area of the bioceramic coating of the coated products—meaning these two variables should fluctuate in relation to each other or each increase or decrease in parallel.

### Confirming Bioceramic Coating Homogeneity & Repeatability Ratio

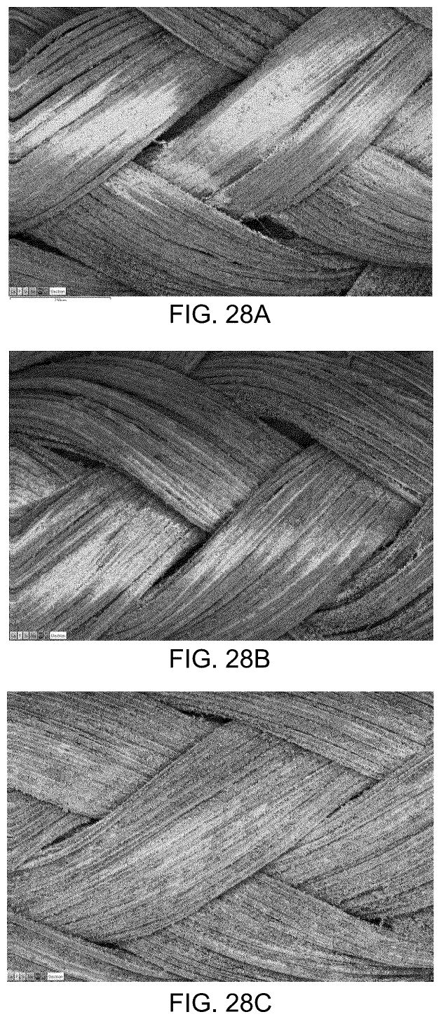

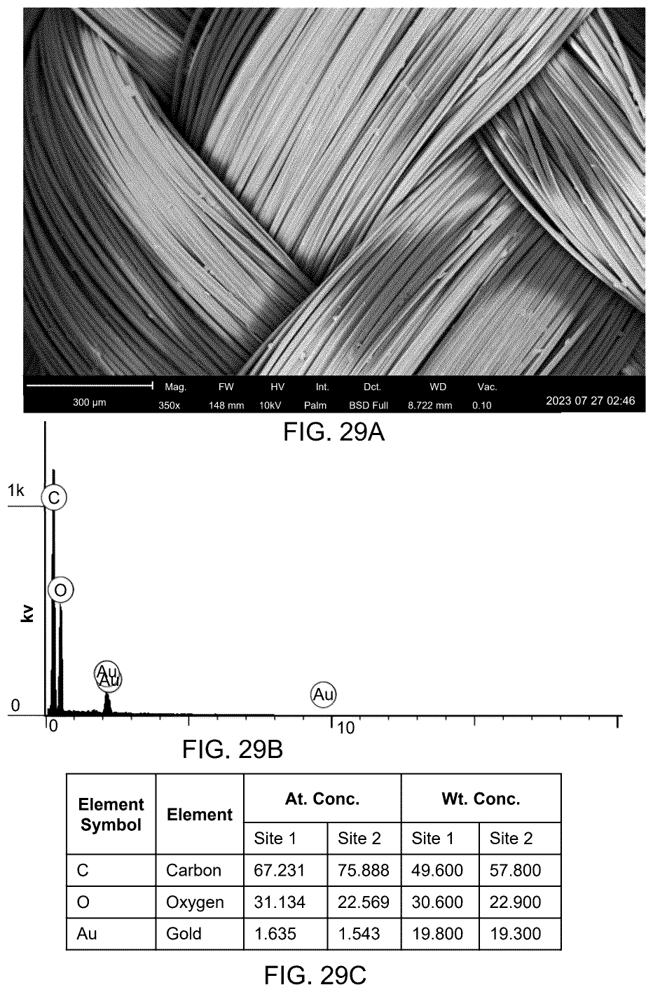

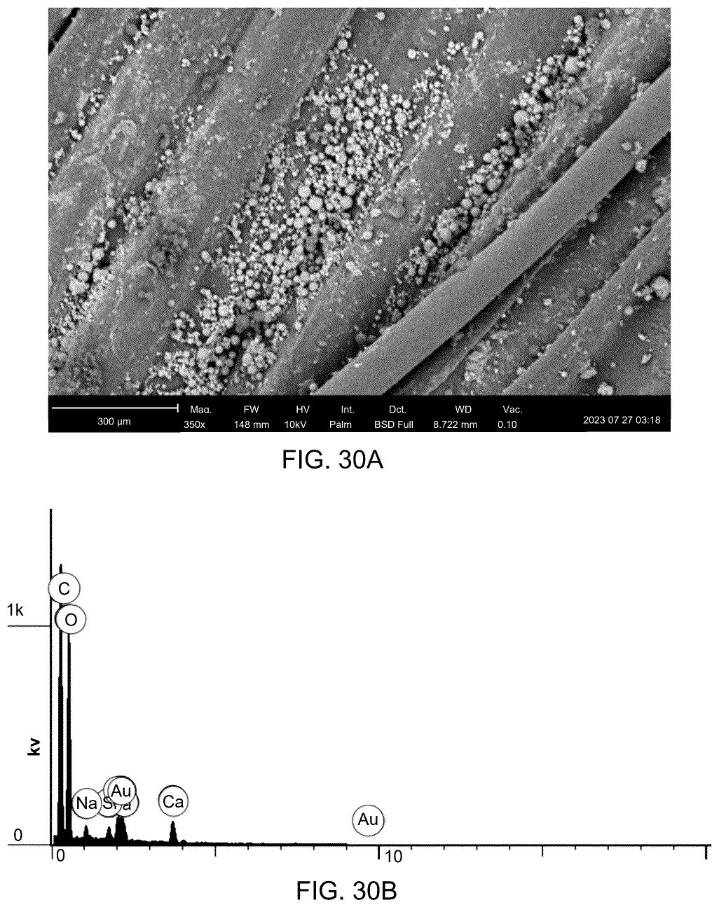

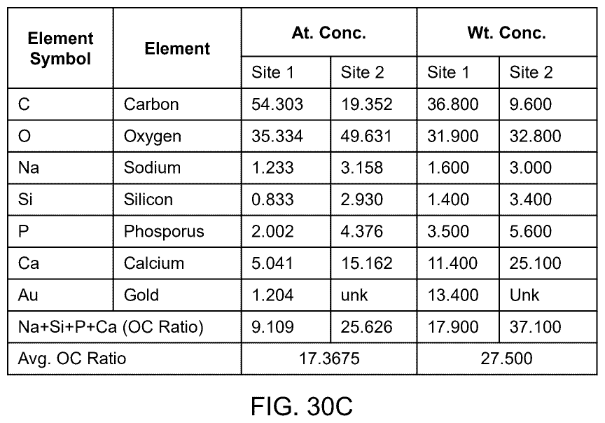

Groups 1 and 2 were sent to an outside laboratory to collect the SEM images, SEM-EDS color maps and SEM-EDS spectrum as shown for FIGS. 28A-28C, 29A-29B (Group 1) and FIGS. 30A-30B (Group 2). At least two site locations on each of Group 1 and 2 were evaluated and shown in tables of FIG. 29C (Group 1) and FIG. 30C (Group 2). The average OC ratio was calculated for Group 2 and expressed in At % and Wt %. The average OC ratio was 17.4 at % and 27.5 wt % respectively. The 27.5 wt % is confirmed and significantly over the set QC criterion of 4.7%.

### EXAMPLE EMBODIMENTS

Claim 1. A coated textile product having an improved bioactive, biological or healing response comprises: a textile, the textile comprising an outer diameter or outer surface and a textile material; a first coating, the first coating disposed over a portion of the of the outer surface or outer diameter, the first coating comprising a first coating material and a first coating thickness; and a second coating, the second coating disposed over a portion of the first coating, the second coating comprising a second coating material and a second coating thickness, the second coating includes a bioceramic coating.

Claim 2. The coated textile product of claim 1, wherein the first coating comprises a binder coating.

Claim 3. The coated textile product of claim 2, wherein the binder coating comprises Bionate.

Claim 4. The coated textile product of claim 1, wherein the bioceramic material comprises bioactive or bioinert bioceramic material.

Claim 5. The coated textile product of claim 4, wherein the bioactive bioceramic material comprises Bioglass.

Claim 6. The coated textile product of claim 5, wherein the Bioglass comprises the molecules Si, Ca, Na and P.

Claim 7. The coated textile product of claim 1, wherein the textile comprises a suture, a soft anchor, or a textile graft.

Claim 8. The coated textile product of claim 1, wherein the textile comprises textile construction, the textile structure includes a woven structure, a braided structure or a knitted structure.

Claim 9. The coated textile product of claim 1, wherein the textile material comprises a biocompatible and non-biodegradable polymer.

Claim 10. The coated textile product of claim 1, wherein the textile material comprises a biocompatible and biodegradable polymer.

Claim 11. The coated textile product of claim 10, wherein the biodegradable polymer comprises semi-synthetic or synthetic polymers.

Claim 12. The coated textile product of claim 11, wherein the semi-synthetic polymers comprises a protein derivate, a polysaccharide derivates, a protein, a polysaccharide (e.g., cellulose), and/or any combination thereof.

Claim 13. The coated textile product of claim 11, wherein the synthetic polymers comprise polyolefins, vinyl polymers, fluoropolymers, polyesters, polyamides, polysulfones, polyacrylics, polyacetals, polyimides, polycarbonates, polyurethanes and/or any combination thereof.

Claim 14. The coated textile product of claim 1, wherein the bioceramic material comprising one or more ceramic particles, the one or more ceramic particles including a particle size of 0.01 to 10 micrometers (μm).

Claim 15. The coated textile product of claim 1, wherein the first coating thickness and the second coating thickness comprises at least 0.01 μm or greater.

Claim 16. The coated textile product of claim 1, wherein the second coating thickness is less than the first coating thickness.

Claim 17. The coated textile product of claim 1, wherein the first coating thickness is less than the second coating thickness.

Claim 18. The coated textile product of claim 1, wherein the first coating thickness is 50% smaller than the second coating thickness.

Claim 19. The coated textile product of claim 1, wherein the first coating thickness is 50% smaller than an average particle size of the second coating.

Claim 20. The coated textile product of claim 1, wherein the bioceramic coating comprises one or more ceramic particles and a solvent.

Claim 21. The coated textile product of claim 1, wherein the bioceramic coating comprises one or more ceramic particles and a non-solvent.

Claim 22. The coated textile of claims 20 and 21, wherein the one or more ceramic particles comprises a particle size, the particle size comprises at least 0.01 μm or greater.

Claim 23. The coated textile product of claim 20, wherein the solvent comprises at least 2% Volume of solvent or greater.

Claim 24. The coated textile product of claim 20 or 21, wherein the one or more ceramic particles comprises a concentration, the concentration comprises at least 2% mass of ceramic particles.

Claim 25. The coated textile product of claim 1, wherein the second coating comprises a percent change of weight (% wt), the % wt comprises at least a 0.1% mass change of weight.

Claim 26. The coated textile product of claim 1, wherein the coated textile product comprises a homogeneity ratio that predicts enhanced or optimized healing response, the homogeneity ratio comprises less than or equal to 10%.

claim 27. The coated textile product of claim 1, wherein the second coating disposed over a portion of the first coating comprises a disposition on an outer surface of the first coating.

Claim 28. The coated textile product of claim 1, wherein the second coating disposed over a portion of the first coating comprises a disposition that is partially embedded.

Claim 29. The coated textile product of claim 1, wherein the second coating disposed over a portion of the first coating comprises a disposition that is fully embedded.

Claim 30. The coated textile product of claim 1, wherein first coating comprises one or more first coating particles, the one or more first coating particles comprises a first coating particle size, the first coating particle size comprises at least 0.01 μm or greater.

Claim 31. A method to characterize a coated textile product that will have an enhanced or optimized healing response, comprises the steps of: predefining one or more quality control (QC) acceptance criteria that correlates or predicts one or more coated products having an improved or optimized healing response; selecting a sample, portion, subset or fraction of the one or more coated products; characterizing a sample, portion, subset or fraction of one or more coated products using one or more characterization techniques to collect/extract/acquire QC sample control data; confirming qc sample control data meets or substantially meets qc acceptance criteria; and passing or approving one or more coated products with an improved or successful biological response.

Claim 32. A method to characterize a coated textile product that will have an enhanced or optimized bioactive or biocompatible healing response, comprises the steps of: providing one or more coated products; selecting a sample, portion, subset or fraction of the one or more coated products; characterizing a sample, portion, subset or fraction of one or more coated products using one or more characterization methods to collect QC sample control data; confirming or comparing the qc sample control data of the sample, portion, subset or fraction of the one or more coated products satisfies one or more QC acceptance criteria prior to acceptance of the one or more coated products; and approving the accepted one or more coated products that comprises an optimized or enhanced biological response.

Claim 33. The method of claim 31, wherein the step of predefining one or more quality control (QC) acceptance criteria that correlates or predicts one or more coated products having an improved or optimized healing or bioactive response comprises the steps of: providing one or more coated products; characterizing the one or more coated products using one or more characterization methods to collect raw data; and generating one or more QC acceptance criteria that predicts or correlates the one or more coated products having improved or optimized bioactivity and/or biocompatible response (e.g., healing response).

Claim 34. The method of claims 31 to 33, the one or more characterization methods comprise the collection of chemical, physical, mechanical properties or healing response or biological response properties.

Claim 35. The method of claims 31 to 33, the one or more characterization methods comprise the collection of raw data that includes an average or median coating wall thickness, % weight of coating or coating weight increase, coating particle size, identification of coating molecules, identification of atomic weight of each of the coating molecules, coating uniformity, coating roughness, coating adherence, coated product flexibility, coated product elongation, coated product knot breakage, coated product accelerated aging, coated product shelf-life, implantation pull out strength, implantation geometry, implantation bone reactions, implantation tissue reactions, implantation osteointegration, implantation inflammatory biomarkers, implantation pH, or implant bone tunnel diameter.

Claim 36. The method of claims 31 to 33, the QC acceptance criteria comprises: % weight of coating increase, coating particle size, identification of atomic weight of each of the coating molecules, coating uniformity, coating adherence, homogeneity ratio and/or any combination thereof.

Claim 37. The method of claims 32 and 33, wherein the optimized healing or bioactive response comprises increased osteointegration, the osteointegration includes properties having increased implantation pull-out strength, reduced or decreased implantation bone reactions, decreased implantation tissue reactions, decreased inflammatory biomarkers, decreased implantation pH, decreased bone tunnel diameter, decreased migration, decreased implant degradation, and/or any combination thereof.

Claim 38. The method of claim 32 or 33, wherein the step of providing one or more coated products comprises a textile, a first coating layer and a second coating layer.

Claim 39. The method of claim 38, wherein the first coating layer comprises a binder coating and the second coating layer comprises a bioceramic coating.

Claim 40. The method of claim 39, wherein the bioceramic coating comprises the molecules of Si, Na, Ca and P.

Claim 41. The method of claim 40, wherein the molecules of Na, Ca, Si and P comprise at least an atomic weight % of 5% or greater and/or a weight % of 5% or greater.

Claim 42. The method of claim 34, wherein the collection of chemical properties comprises the identification of molecules in the second coating.

Claim 43. The method of claim 35, wherein the homogeneity ratio equals the sum of atomic % of Ca+Na+P+Si or sum of weight % of Ca+Na+P+Si.

Claim 44. The method of claim 35, wherein the homogeneity ratio equals the sum of atomic % of (Ca+Na+P+Si) divided by N or the sum of weight % of (Ca+Na+P+Si) divided by N.

Claim 45. The method of claim 39, wherein the bioceramic coating comprises a first one or more ceramic particles and a second one or more ceramic particles.

Claim 46. The method of claim 38, wherein the second coating layer comprises a dispersion, the dispersion includes a first one or more ceramic particles, a second one or more ceramic particles, and a treating solvent.

Claim 47. The method of claim 46, wherein the treating solvent comprises a solvent and a non-solvent.

Claim 48. The method of claims 45 and 46, wherein the first one or more ceramic particles comprises Bioglass and the second one or more ceramic particles comprises HAp.

Claim 49. The coated textile product of claim 1, wherein the first or second coating comprises at least one metal ion.

Claim 50. The method of claims 32 and 33, wherein the optimized healing or bioactive response comprises an antimicrobial response.

Claim 51. The method of claims 32 and 33, wherein the optimized healing or bioactive response comprises osteointegration and antimicrobial response.

Claim 52. The method of claim 51, wherein the antimicrobial response comprises an antimicrobial property having an bioactive glass particle size, the bioactive glass particle size comprises at least 10 μm or greater.

Claim 53. A bioceramic coated product comprises: a textile product, the textile product comprising an outer surface and a product material, the product material includes a biocompatible, nonbiodegradable polymer; a first coating layer, the first coating layer comprising a first coating material and a first solvent, the first coating layer disposed over a portion of the outer surface of the textile product, the first coating material includes a biocompatible, nonbiodegradable polymer; and a second coating layer, the second coating layer disposed over a portion of the first coating, the second coating layer comprising one or more bioceramic particles and a second solvent, at least a portion of the one or more bioceramic particles is positioned below the first coating; and a bioceramic coated product homogeneity OC ratio, the bioceramic coated product homogeneity OC ratio is greater than or equal to a quality control OC ratio of 4.7 wt. %.

Claim 54. The bioceramic coated product of claim 53, wherein the textile product comprises a suture or suture anchor.

Claim 55. The bioceramic coated product of claim 54, wherein biocompatible, nonbiodegradable polymer of the product material comprises PET or UHWMPE.

Claim 56. The bioceramic coated product of claim 53, wherein biocompatible, nonbiodegradable polymer of the first coating comprises Bionate.

Claim 57. The bioceramic coated product of claim 53, wherein the one or more bioceramic particles comprises Bioglass.

Claim 58. The bioceramic coated product of claim 53, wherein the one or more bioceramic particles comprises Bioglass and HA.

Claim 59. The bioceramic coated product of claim 57 or 58, wherein the Bioglass comprises a plurality of molecules, the plurality of molecules includes Na, Ca, P and Si.

Claim 60. The bioceramic coated product of claim 59, wherein the bioceramic coated product homogeneity ratio equals the sum of the wt % of the plurality of molecules of the one or more bioceramic particles of the second coating layer acquired by Energy Dispersive X-Ray Spectroscopy (EDS) on at least one predetermined location, n, along a length of the bioceramic coated textile product and divided by n.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

As previously noted, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

I/We claim:

1. A method of manufacturing a repeatable, homogenously bioceramic coated textile product, comprises the steps of:
Depositing a first coating layer onto a portion of a textile product, the first coating layer comprises a first solvent and a first material, the first material includes a biocompatible, nonbiodegradable polymer;
Depositing a second coating layer onto the first coating layer, the second coating layer comprises a second solvent and a second coating material, the second coating material includes one or more bioceramic particles having a plurality of molecules comprising at least Sodium (Na), Phosphorus (P), Silicone (Si) and Calcium (Ca);
Removing the second solvent from the second coating layer to enable at least a portion of the one or more bioceramic particles to be positioned below an outer surface of the first coating layer; and
Verifying that the coated textile product comprises a homogeneous second coating layer by meeting or exceeding the homogeneity osteoconductive (OC) ratio of 4.7 wt %,
wherein the homogeneity OC ratio equals the sum of the wt % of the plurality of molecules of the one or more bioceramic particles of the second coating layer acquired by Energy Dispersive X-Ray Spectroscopy (EDS) on at least one predetermined location, n, along a length of the bioceramic coated textile product and divided by n.

2. The method of manufacturing of claim 1, wherein the textile product comprises a biocompatible, nonbiodegradable polymer.

3. The method of manufacturing of claim 1, wherein the textile product comprises a suture or a suture anchor.

4. The method of manufacturing of claim 1, wherein the one or more bioceramic materials comprises Bioglass® and hydroxyapatite (HA).

5. The method of manufacturing of claim 1, wherein the textile product comprises a suture or a suture anchor.

6. The method of manufacturing of claim 1, wherein the biocompatible, nonbiodegradable polymer of the first material comprises a thermoplastic elastomer.

7. The method of manufacturing of claim 1, wherein the OC ratio of 4.7 wt % produces a homogeneously coated product that will have a consistent in-vivo bioactive healing response.

8. The method of manufacturing of claim 7, wherein the bioactive healing response comprises equal to or superior osteointegration, antimicrobial, and angiogenesis response than uncoated textile products.

9. The method of characterizing a homogeneously coated textile product, comprising the steps of:
manufacturing at least one coated textile product; the at least one coated textile product comprises:
a. a textile product, the textile product comprising an outer surface and a product material, the product material includes a biocompatible, nonbiodegradable polymer;
b. a first coating, the first coating comprising a first coating material, the first coating disposed over a portion of the outer surface of the textile product, the first coating material includes a biocompatible, nonbiodegradable polymer; and
c. a second coating, the second coating disposed over a portion of the first coating, the second coating comprising one or more bioceramic particles, at least a portion of the one or more bioceramic particles is positioned below an outer surface of the first coating, the one or more bioceramic particles includes a plurality of elements of at least Sodium (Na), Phosphorus (P), Silicone (Si) and Calcium (Ca);
collecting the wt % of each of the plurality of elements of the one or more bioceramic particles of the second coating from at least one predetermined location on the at least one coated textile product using Energy Dispersive X-Ray Spectroscopy (EDS);
determining the homogeneity OC ratio by summing the wt % of each of the plurality of elements of the second coating and dividing by four; and
confirming the homogeneity OC ratio meets or exceeds 4.7 wt %,
wherein the homogeneity OC ratio of 4.7 wt % or greater produces a homogeneously coated textile product with a consistent in-vivo bioactive response.

10. The method of characterizing of claim 9, wherein the biocompatible, nonbiodegradable polymer comprises PET or UHWMPE.

11. The method of characterizing of claim 9, wherein the textile product comprises a suture or a suture anchor.

12. The method of characterizing of claim 9, wherein the consistent in-vivo bioactive response comprises osteogenesis, antimicrobial and angiogenesis responses.

13. A method of developing quality control (QC) acceptance criteria to manufacture bioceramic coated products having homogenous bioceramic coating comprises the steps of:
obtaining at least one bioceramic coated product, the at least one bioceramic coated product comprising one or more bioceramic particles, the one or more bioceramic particles comprising a plurality of elements that include Sodium (Na), Phosphorus (P), Silicone (Si) and Calcium (Ca);
characterizing the at least one bioceramic coated product by collecting the wt. % of each of the plurality of elements of the one or more bioceramic particles from at least one predetermined location on the at least one bioceramic coated textile product using Energy Dispersive X-Ray Spectroscopy (EDS);
determining an average product homogeneity OC ratio by summing the wt % of each of the plurality of elements of the one or more bioceramic particles of the at least one bioceramic coated product and dividing by the at least one predetermined location number; and
comparing a QC homogeneity OC ratio to the average product homogeneity OC ratio; and
approving the at least one bioceramic coated product if the average product homogeneity OC ratio is equal to or greater than the QC homogeneity OC ratio.

14. The method of developing of claim 13, wherein the at least one bioceramic coated product further comprises at least one bioceramic coated textile product.

15. The method of developing of claim 14, wherein the textile product is a suture or suture anchor.

16. The method of developing of claim 13, wherein the QC homogeneity OC ratio equals 4.7 wt. %.
